(19) United States
(12) Patent Application Publication
(10) Pub. No.: US 2005/0084914 A1
(43) Pub. Date: Apr. 21, 2005

(54) Assays with primary cells

(76) Inventors: J. Gordon Foulkes, Encinitas, CA (US); Oren E. Beske, Sunnyvale, CA (US); Simon Goldbard, San Jose, CA (US)

Correspondence Address: KOLISCH HARTWELL, P.C., 520 S.W. YAMHILL STREET, SUITE 200, PORTLAND, OR, 97204, US

(21) Appl. No.: 10/942,322

(22) Filed: Sep. 15, 2004

Related U.S. Application Data
(60) Provisional application No. 60/503,406, filed on Sep. 15, 2003.

(51) Int. Cl. G01N 33/53; G01N 33/567

(52) U.S. Cl. 435/7.2

(57) ABSTRACT

Systems, including methods, apparatus, compositions, and kits, for performing assays with primary cells.

### CROSS-REFERENCE TO PRIORITY APPLICATION

This application is based upon and claims the benefit under 35 U.S.C. § 119(e) of the following U.S. provisional patent application, which is incorporated herein by reference in its entirety for all purposes: Ser. No. 60/503,406, filed Sep. 15, 2003.

### CROSS-REFERENCES TO RELATED APPLICATIONS

This application incorporates by reference in their entirety for all purposes the following U.S. patent applications: Ser. No. 09/549,970, filed Apr. 14, 2000; Ser. No. 09/694,077, filed Oct. 19, 2000 Ser. No. 10/120,900, filed Apr. 10, 2002; Ser. No. 10/238,914, filed Sep. 9, 2002; Ser. No. 10/273,605, filed Oct. 18, 2002; Ser. No. 10/282,904, filed Oct. 28, 2002; Ser. No. 10/282,940, filed Oct. 28, 2002; Ser. No. 10/382,796, filed Mar. 5, 2003; Ser. No. 10/382,797, filed Mar. 5, 2003; Ser. No. 10/382,818, filed Mar. 5, 2003; Ser. No. 10/407,630, filed Apr. 4, 2003; Ser. No. 10/444,573, filed May 23, 2003; Ser. No. 10/445,291, filed May 23, 2003; Ser. No. 10/713,866, filed Nov. 14, 2003; Ser. No. 10/842,954, filed May 10, 2004; and Ser. No. 10/901,942, filed Jul. 28, 2004.

This application also incorporates by reference in their entirety for all purposes the following U.S. provisional patent applications: Ser. No. 60/129,664, filed Apr. 15, 1999; Ser. No. 60/170,947, filed Dec. 15, 1999; Ser. No. 60/241,714, filed Oct. 18, 2000; Ser. No. 60/259,416, filed Dec. 28, 2000; Ser. No. 60/293,863, filed May 24, 2001; Ser. No. 60/299,267, filed Jun. 18, 2001; Ser. No. 60/299,810, filed Jun. 20, 2001; Ser. No. 60/307,649, filed Jul. 24, 2001; Ser. No. 60/307,650, filed Jul. 24, 2001; Ser. No. 60/310,540, filed Aug. 6, 2001; Ser. No. 60/317,409, filed Sep. 4, 2001; Ser. No. 60/318,156, filed Sep. 7, 2001; Ser. No. 60/328,614, filed Oct. 10, 2001; Ser. No. 60/343,682, filed Oct. 26, 2001; Ser. No. 60/343,685, filed Oct. 26, 2001; Ser. No. 60/344,482, filed Oct. 26, 2001; Ser. No. 60/344,483, filed Oct. 26, 2001; Ser. No. 60/345,606, filed Oct. 26, 2001; Ser. No. 60/348,025, filed Oct. 26, 2001; Ser. No. 60/348,027, filed Oct. 26, 2001; Ser. No. 60/359,207, filed Feb. 21, 2002; Ser. No. 60/362,001, filed Mar. 5, 2002; Ser. No. 60/362,055, filed Mar. 5, 2002; Ser. No. 60/362,238, filed Mar. 5, 2002; Ser. No. 60/370,313, filed Apr. 4, 2002; Ser. No. 60/383,091, filed May 23, 2002; Ser. No. 60/383,092, filed May 23, 2002; Ser. No. 60/413,407, filed Sep. 24, 2002; Ser. No. 60/413,675, filed Sep. 24, 2002; Ser. No. 60/421,280, filed Oct. 25, 2002; Ser. No. 60/426,633, filed Nov. 14, 2002; Ser. No. 60/469,508, filed May 8, 2003; Ser. No. 60/473,064, filed May 22, 2003; Ser. No. 60/503,406, filed Sep. 15, 2003; Ser. No. 60/523,747, filed Nov. 19, 2003; Ser. No. 60/537,454, filed Jan. 15, 2004; and Ser. No. 60/585,150, filed Jul. 2, 2004.

This application incorporates by reference in its entirety for all purposes the following PCT patent application: Serial No. PCT/US01/51413, filed Oct. 18, 2001, and published as Pub. No. WO 02/37944 on May 16, 2002.

### BACKGROUND

The pharmaceutical industry's search for new drugs typically begins by “screening” compounds from libraries of member compounds for promising biological activity (e.g., preventing or delaying the onset of disease). “Hits,” or compounds that show promise following such primary screening, may be studied further via secondary screening, lead compound screening (or optimization), and clinical evaluation, among others. The screening process in particular has become highly automated, with today's screening instruments capable of screening thousands to tens of thousands of compounds for promising activity in a single day.

Unfortunately, despite this automation, screening assays and subsequent clinical evaluation still have a number of shortcomings. For example, in current screening assays, library compounds are tested for activity against “biological” systems that may differ significantly from the ultimate target(s) of a drug in a patient. These biological systems may include isolated targets, such as purified enzymes or receptors, acting on synthetic or model substrates. These biological systems also may include cell lines that are immortalized and/or transformed, often obtained from nonhuman species.

These and/or other shortcomings may be addressed at least in part by using more “physiological” assays. For example, such assays could employ primary cells, rather than isolated reaction components or established cell lines. Primary cells are cells that have been obtained recently from humans or other species, generally without subsequent immortalization or transformation, although they may be cultured. Primary cells are, by their nature, a more accurate representation of human subjects than the established cell lines currently used in drug screening. Unfortunately, primary cells are difficult to obtain, maintain, and use, with their limited numbers generally making it prohibitively expensive (or impossible) to use these cells in multiple assays. Thus, there is a need for technology that could increase the use of primary cells by obtaining relevant information from smaller numbers of cells.

### SUMMARY

The present teachings provide systems, including methods, apparatus, compositions, and kits, for performing assays with primary cells.

### BRIEF DESCRIPTION OF THE DRAWINGS

FIG. 1 is a flowchart showing configurations of primary cells, carriers, vessels, and potential modulators, among others, produced before, during, and/or after performing steps of an exemplary method of assaying primary cells connected to carriers, in accordance with aspects of the present teachings.

FIG. 2 is a somewhat schematic representation of the effect of potential modulators on primary cells measured from a configuration created by the exemplary method of FIG. 1.

FIG. 3 is a flowchart showing method steps that may be included in an exemplary method of assaying primary cells using particles, in accordance with aspects of the present teachings.

FIG. 4 is a flowchart showing configurations produced before, during, and/or after performing steps of an exemplary method of multiplexed assay of primary cells connected to coded carriers, in accordance with aspects of the present teachings.

FIG. 5 is a flowchart showing an exemplary method using coded carriers and primary cells to stratify patients and/or modulators in vitro prior to clinical trials.

FIG. 6 is a graph showing data for the proliferative response of primary (HRPTE) cells to camptothecin, obtained generally according to the method of FIG. 5.

FIG. 7 is a graph showing data for the cytotoxicity response of primary (HRPTE) cells to staurosporine, obtained generally according to the method of FIG. 5.

FIG. 8 is a graph showing the results of adipocyte differentiation assays induced with a cocktail of inducers or no inducer and performed with a relatively large number of cells disposed in separate wells of a standard 96-well microplate.

FIG. 9 is a graph showing the results of adipocyte differentiation assays induced with a cocktail of inducers or no inducer and performed by multiplexed analysis using coded carriers and with about 50-fold fewer cells than the assay of FIG. 8, in accordance with aspects of the present teachings.

FIG. 10 is a graph showing the results of adipocyte differentiation assays induced with troglitazone, based on lipid accumulation and performed on sets of primary cells from four human subjects by using coded carriers for assay of the sets in a shared fluid volume, in accordance with aspects of the present teachings.

FIG. 11 is a graph showing the results of PPARγ activity assays induced with troglitazone, based on PEPCK expression, and performed on sets of primary cells from the same human subjects as FIG. 10, using coded carriers for assay of the sets in a shared fluid volume, in accordance with aspects of the present teachings.

FIG. 12 is a graph showing the results of PPARγ activity assays comparing the effects of different PPARγ agonists on primary cells from the same human subjects as FIG. 10, using coded carriers for assay of the sets in a shared fluid volume, in accordance with aspects of the present teachings.

### DETAILED DESCRIPTION

The present teachings provide systems, including methods, apparatus, compositions, and kits, for performing assays with primary cells. The systems may place the primary cells in a locally concentrated or clustered configuration (with a higher degree of confluence) within an assay compartment, for example, a higher density of the cells on a subset of the support surfaces of the compartment. The subset of support surfaces may be preselected and may be provided by one or more carriers disposed in the compartment and/or by a separately addressable sub-compartment within the compartment. In some examples, the carriers may be distinguishable so that two or more sets of primary cells may be assayed together in the same volume of fluid (a shared volume). Placing the primary cells in a smaller area at a higher density may provide a variety of benefits, for example, allowing the assay of relatively small numbers of physiologically relevant primary cells.

The systems of the present teachings may provide dramatic improvements in the use of primary cells. Primary cells thus may be used to screen compounds and/or human subjects, for example, to prioritize potential drugs for further study and/or to stratify human subjects for identification of better candidates for treatment with one or more, of the compounds, among others. In the past, the limited numbers of primary cells generally made it prohibitively expensive (and technically impractical or impossible) to test the activity of a lead compound (and particularly a set of lead compounds) on primary cells from a relatively large group of human subjects. Accordingly, there were no in vitro screening systems available that provided a feasible, physiologically relevant approach to prioritizing lead compounds according to their expected effectiveness within the population. As a result, without meaningful criteria for in vitro prioritization, lead compounds that produce a substantial variability of response within the population were selected for clinical trials, resulting in drugs with a low frequency of effectiveness within the population and thus unpredictable benefit to the patient. Furthermore, the lack of meaningful in vitro screening systems prevented routine selection of a drug best suited for administration to a particular human subject based on the response of the subject's cells in vitro to a relatively large panel of candidate drugs. The systems of the present teachings may provide a more physiologically meaningful and clinically relevant approach to screening in vitro than assays with established cell lines.

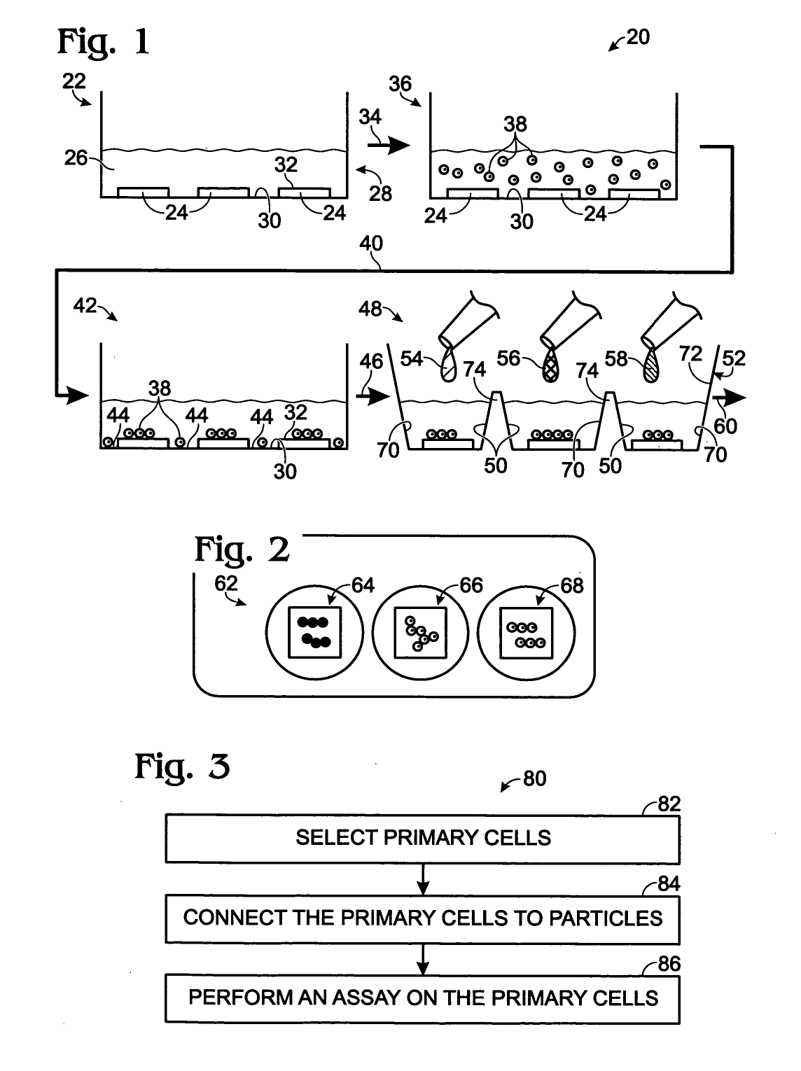

FIG. 1 shows a flowchart 20 illustrating configurations produced before, during, and/or after performing steps of an exemplary method of assaying small numbers of primary cells using carriers. The carriers may be configured to be relatively small, discrete particles. Due to a small size and thus small surface area, each particle can hold a relatively small number of cells at a relatively high density adjacent one or more surface regions of the particle (generally, in, on, and/or about the particle). This high density may be sufficient to keep the cells alive and in a physiological condition suitable for assays. Alternatively, or in addition, cells may be positioned and/or assayed in discrete subcompartments, which may be brought into and/or out of fluid communication, as described below.

Relevant materials may be selected and combined to form a first configuration 22 (shown in the top left panel of FIG. 1). Here, particles 24 are disposed in a fluid 26 and supported by a vessel 28. The particles may be generally planar, as presented here, or may have any other suitable geometry (or geometries), such as spherical, ovoid, cubical, cylindrical, etc. The particles may be arrayed on a support surface 30 of vessel 28, so that a planar face 32 of each particle faces upward. This planar surface (or any other suitable exterior/interior surface region(s) of each particle) may define an attachment region for connecting cells and/or a detection area used for measuring signals from the cells.

Primary cells may be added to fluid in the vessel, shown at 34, to produce a second configuration 36 (shown in the top right panel of FIG. 1). Primary cells 38 may be human cells isolated from a human subject. In some examples, the cells may be isolated from a disease patient having a disease phenotype and/or may be from an unaffected or less-affected tissue of a control or disease patient lacking the disease phenotype (that is, having a normal or nondiseased phenotype). The cells may be aggregated or non-aggregated, as shown in the present illustration, when added to the vessel.

The cells may be connected to the particles, shown at 40, to produce a third configuration 42 (shown in the bottom left panel of FIG. 1). Connection to the particles may involve contact of the cells with the particles, particularly planar face 32, such as through settling or sedimentation driven by gravity or centrifugation. Accordingly, cells may settle onto the top surfaces of the particles and between the particles, onto the support surface 30 of the vessel. By substantially covering the support surface of the vessel with particles, a majority of the cells placed into the vessel may settle onto and connect to particles, thus maximizing the use of the cells. Alternatively, or in addition, the particles may be moved to facilitate contact with the cells, such as by stirring or agitating the fluid, placing the particles onto cells, and/or the like. The particles may form any suitable connection to the primary cells, including covalent and/or noncovalent connections, generally to resist separation of the particles and cells, at least until an assay has been initiated.

The cells may be disposed in relatively close proximity on the particles at this stage and/or when signals are detected from the cells. For example, the cells may cover at least about 25%, 50%, or 75%, among others, of an attachment region and/or detection area of the particles. In some examples, at least about one-half of the cells may be spaced from another cell (as measured at a position of closest cell-cell approach) by less than the average diameter of the cells, and/or may be in contact with another cell. In some examples, the density of the cells may be about the same on the particles and on adjacent, exposed surface regions 44 of the vessel support surface 30 until the particles are moved to assay compartments.

The cells and their connected particles may be transferred to two or more separate compartments, shown at 46, to produce a fourth configuration 48 (shown in the bottom right panel of FIG. 1). The compartments may be defined, for example, by wells 50 of a microplate 52, among others. One or more particles may be transferred to each compartment. The particle(s) and their connected cells in each compartment may be exposed to a different potential modulator (e.g., a modulator of known or unknown clinical effect), for example, contacted with different chemical compounds 54, 56, 58. In some embodiments, the particles may be transferred to separate vessels by other mechanisms, such as flow sorting, among others. In this fourth configuration, the cells connected to the particles may be disposed at a substantially higher density adjacent (in, on, and/or about) the particles than adjacent (in, on, and/or about) the entire support surface on which the particles are resting. The density of the cells adjacent the entire support surface is determined as a density averaged over the entire support surface.

An effect, if any, of each potential modulator on the cells may be detected, shown at 60. Detection may include collecting data (sensing signals) from the cells, associated regions of the particles, and/or other regions of the compartment in which the cells are disposed (such as surrounding fluid or compartment surfaces, among others), to determine an effect on the cells. Data collection may be performed spectroscopically, by imaging, etc. Detection also may include analyzing the data to determine if the potential modulator affected the cells, for example. Such data analysis may be performed automatically, that is, with a controller (such as a computer), and/or by a person.

FIG. 2 shows exemplary image data 62 that may be produced by contacting the primary cells with modulators 54, 56, 58. In particular, in the present illustration, cell sample 64 exposed to modulator 54 (see FIG. 1) shows an effect on the cells (indicated here by shading) not exhibited by exposure of cell samples 66 and 68 to modulators 56 and 58, respectively.

In some exemplary methods, primary cells may be assayed in divided assay compartments that permit adjustable fluid communication. Divided assay compartments may be used with or without carriers, that is, cells may be directly connected to support surfaces of the divided compartments or connected to carriers placed in the divided compartments.

Configuration 48 of FIG. 1 illustrates an exemplary structure for divided assay compartments, or sub-compartments 70, that may be included in a compartment 72 of microplate 52. Sub-compartments 70 may be addressable in fluidic isolation, as shown in configuration 48, or in fluidic communication by adding fluid to one or more of the sub-compartments until the level of the fluid is above walls 74 that separate the sub-compartments. Accordingly, sets of primary cells may be placed in different sub-compartments with the sub-compartments in fluidic isolation, and then treated together with modulators/reagents with the sub-compartments in fluidic communication (and/or treated separately in fluidic isolation). These sets of primary cells may be placed into the sub-compartments directly and/or in association with a particle and/or other support. Further aspects of sample holders that permit adjustable fluid communication and methods of using these sample holders for cell assays are included in the following patent applications, which are incorporated herein by reference: U.S. patent application Ser. No. 10/282,940, filed Oct. 28, 2002; and U.S. Provisional Patent Application Ser. No. 60/585,150, filed Jul. 2, 2004.

FIG. 3 is a flowchart showing method steps that may be included in an exemplary method 80 of assaying primary cells using particles, in accordance with aspects of the present teachings. The method steps may be performed in any suitable combination, in any suitable order, and any suitable number of times. Primary cells may be selected, shown at 82. Further aspects of primary cells and selection of primary cells are included below, for example, in Sections I and VII. The primary cells may be connected to particles, shown at 84, or may be used in assays without connection to particles, as described above. Further aspects of connection of primary cells to particles are described below, for example, in Section VIII. An assay may be performed on the primary cells, shown at 86. The assay may be started after the primary cells are connected to the particles. In some cases, the assay may be started after the cells and particles have been transferred to suitable sample holders, such as wells of a microplate(s). In some cases, the assay may include exposing the cells to one or more potential modulators, detecting an effect (or no effect) of the modulators on the cells, and selecting a subset of the potential modulators for further study. Further aspects of assays that may be performed on primary cells and how these assays may be performed are described below, for example, in Section VI and elsewhere in the present teachings. Further aspects of modulators, including the modulators themselves, detecting the effects of the modulators, and selecting modulators (or human subjects) based on the detected effects, are included, for example, in Sections IV, XI, and XII, respectively, and elsewhere in the present teachings.

The present teachings include, but are not limited to, the use of primary cells in connection with apparatus, methods, compositions, or kits disclosed and/or claimed in any or all of the patent applications identified above in the Cross-References and incorporated herein by reference. Exemplary apparatus may include primary cells, obtained from different portions (e.g., tissues or organs) of the same human subject, and/or from the same or different portions of different human subjects, associated with any suitable carrier(s), such as coded carriers. The methods further may include mixing coded carriers supporting different sets of primary cells (including, in some cases, mixtures of primary cells with other cells) in a common (shared) volume of fluid, and/or constructing a database from results obtained from different sets of primary cells and/or potential modulators. The code on the coded carriers may be used to identify any suitable aspect of the associated sets of cells, such as a person(s) and/or tissue from which each set of cells was isolated, cell type, density, number, state, and/or other characteristic(s). The detected response or effect may include, among others, an interaction, such as binding, and/or an activity, such as enzyme activity, and/or a phenotypic response, such as a change in the composition, morphology, behavior, signaling, subcellular distribution, transcription, translation, activity, and/or the like, of primary cells or their components.

The systems disclosed herein are well suited to the study of small numbers of cells. For example, the technology allows (although it does not require) assays to be miniaturized, and it permits the handling and transfer of small aliquots of primary cells into standard microplates or other suitable sample holders. The technology also may be used to provide a microgrowth environment that allows 50 cells or fewer to establish a suitable density or confluency rapidly and be ready for assaying, in some cases without the need for any cell division to increase the number/density of cells before the assay starts (“instant confluency”). This may be important because many cells will not display normal behavior unless they are sufficiently close to other cells, because, for example, many cells have a physiology that depends on signals received from nearby cells. Accordingly, cells that are spaced too far from one another may not show a normal physiology and are thus less likely to provide physiologically relevant data in drug screens. In contrast, the systems of the present teachings may allow the collection of relevant screening data from just a fraction (e.g., 10%, 5%, 1%, or less) of the primary cells that would otherwise be needed for standard assays, with comparable results.

The systems also are well suited to the multiplexed study of multiple sets of primary cells. For example, primary cells of several types obtained from a single human subject (or two or more subjects) may be used for any suitable purpose, such as testing the selectivity of a modulator. Alternatively, or in addition, cells from different human subjects can be used for any suitable purpose, such as testing how successful a drug is in treating a disease phenotype of primary cells in culture, before performing additional studies (such as clinical trials). Alternatively, or in addition, cells from different diseases stages (e.g., normal, primary cancer site cells, and metastatic cells) also can be used for any suitable purpose, such as multiplexed analysis of where a treatment could be more effective. Alternatively, or in addition, primary cell responses can be compared for any suitable purpose with responses from nonprimary (e.g., standardized, transformed) cells and/or other targets, in the same or different assays. Alternatively, or in addition, multiple drug candidates may be tested against multiple patient cells.

The practical implications of this technology are manifold. For example, the technology allows in vitro stratification of patients and/or potential drugs before going into clinical trials. This technology can include, for example, cells from normal, diseased, and/or refractory (i.e., difficult to treat) subjects, among others. In addition, the technology allows the potentially different responses of human subjects to drug leads to be tested first on primary cells and then on human subjects, potentially predicting failures and improving the outcome of lead compounds after clinical testing. Furthermore, the technology can be multiplexed with respect to a variety of parameters, improving the odds of finding effective drugs. Thus, for example, the assays can simultaneously provide a multi-tissue response and/or a multi-patient response, in a primary cell assay, to many leads (e.g., using cells from the tissue of a large target group of patients, such as 100 patients with 10-100 lead compounds). The technology also can be used to assess in vitro cellular toxicology. For example, one or more toxicologically relevant primary cell types and/or established cell lines (e.g., hepatocytes, nephric cell substructures, etc.) may be assayed to predict possible toxicology in human trials, including whether cytotoxicity occurs, and, if so, in what tissues/cell types (and potentially how) it might occur. The technology also can be used to explore mechanism of action, pathway, or multi-tissue evaluation of drug response.

These and other aspects of the present teachings are described in the following sections: (I) overview of an exemplary method of assaying primary cells, (II) primary cells, (III) carriers, (IV) potential modulators, (V) vessels, (VI) detection systems and cell assays, (VII) selecting primary cells, (VIII) connecting primary cells to carriers, (IX) placing primary cells in compartments, (X) exposing cells to potential modulators, (XI) detecting effects of potential modulators, (XII) selecting modulators and/or human subjects based on detected effects, (XIII) databases, and (XIV) examples.

I. Overview of An Exemplary Method of Assaying Primary Cells

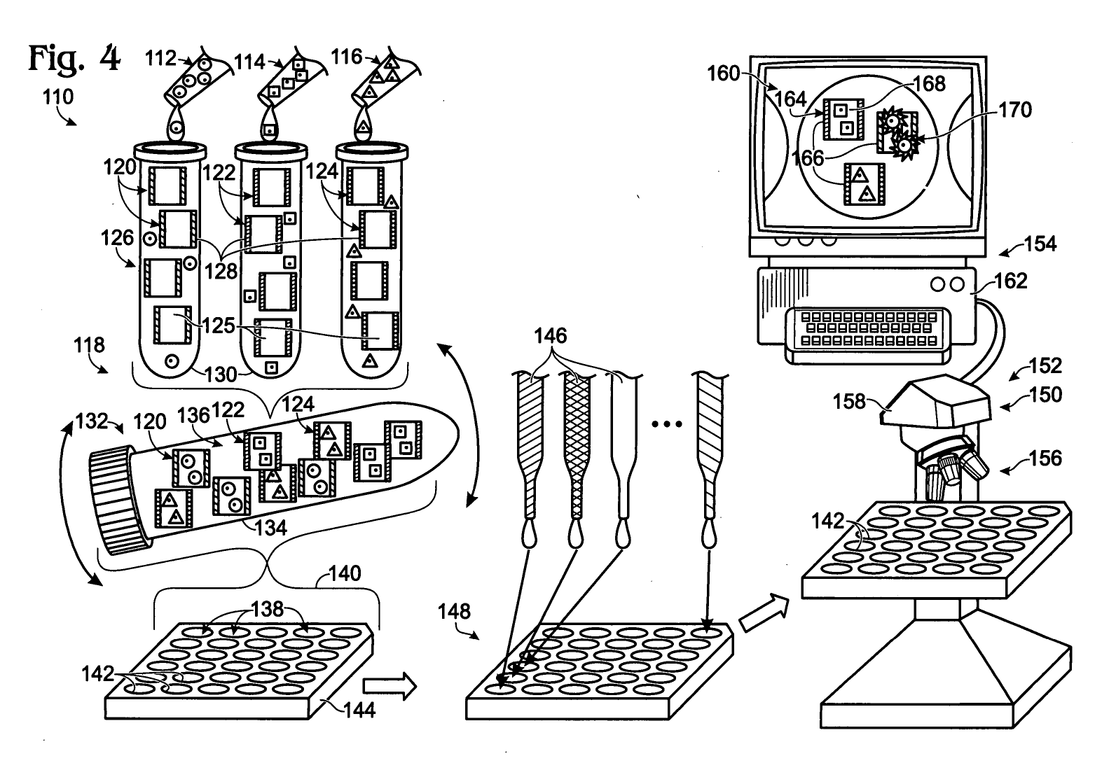

FIG. 4 shows an exemplary method 110 of screening a set of potential modulators for the ability of each modulator to elicit a response (produce an effect) from sets of cells assayed in multiplex. Method 110 may be conducted with two or more sets 112, 114, 116 of primary cells (in this case three). Any suitable primary cells may be selected for use in the method, as described in more detail below in Section VII.

Method 110 may include a step of placing different cell sets 112, 114, 116 in a shared volume of fluid, shown at 118. Even though they are in a shared volume of fluid, the cell sets may be in a segregated configuration, that is, disposed at separate positions within the shared volume, without substantial intermixing of individual cells from different cell sets. Maintaining cells in a segregated configuration enables each cell set to be identified according to a linked code.

The step of placing 118 may include connecting each cell set to a different class (type) 120, 122, 124 of one or more coded carriers 125, shown at 126. Each coded carrier may include a distinguishable code 128 that identifies the cell set connected to the carrier. The step of connecting 126 may be conducted with each class of coded carrier and each corresponding cell set in fluid isolation, such as in vessels 130, to avoid connection of cells from other cell sets to a noncorresponding class of coded carriers.

The step of placing 118 may include mixing the different classes of coded carriers and their connected cell sets, shown at 132. Mixing may be conducted in a vessel, such as a screw-cap tube 134 in the present illustration, or another suitable container. Mixing may include inversion, vortex action, and/or agitation, among others, which may provide a nonpositional mixture 136 in which the different classes of carriers 120, 122, 124 are randomly or arbitrarily distributed relative to one another. Alternatively, different classes of carriers and their connected cell sets may be mixed at an examination site, such as in wells of a microplate.

The step of placing may include dispensing portions of nonpositional mixture 136 to examination sites 138, shown at 140. Each portion may represent each cell set 112, 114, 116 and thus each class of coded carriers 120, 122, 124. Each examination site may be a surface or a vessel, such as a microplate well 142 included in a microplate 144. Further aspects of dispensing carriers are included elsewhere in the present teachings (such as Section IX) and in the patent applications listed above in the Cross-References, which are incorporated herein by reference, particularly U.S. patent application Ser. No. 10/901,942, filed Jul. 28, 2004.

Method 110 may include exposing cell sets 112, 114, 116 (or members thereof) to different potential modulators 146, shown at 148. Exposure to the potential modulators may be performed by addition of a chemical and/or biological agent, such as a test compound, to each well, and/or by treatment with a physical condition. Each cell set may be exposed to each potential modulator for any suitable amount of time. Further aspects of potential modulators and exposure to potential modulators are included, for example, in Sections IV and X and elsewhere in the present teachings.

Method 110 may include detecting effects, shown at 150, produced by exposure of the different cell sets to each potential modulator. Detecting an effect (a response) may be conducted with the cell sets in microplate wells 142, among others. Effects may be measured with any suitable detection system, such as a sensor apparatus (e.g., image capture apparatus 152) to collect data about the cells, and a controller (e.g., image analysis system 154) to process the data. The image capture apparatus may include optics 156, such as those provided by a microscope 158 and a sensor assembly, such as a CCD array or a digital camera. The image capture apparatus may image a field of view from the examination site to collect image data corresponding to at least one image 160 of the coded carriers and the cell sets. In some embodiments, the image may be two or more images produced from the same field of view with different optics, for example, with different filters, or with a different light source or sensor configuration.

Image analysis system 154 may be configured to process the image data, for example, to extract information from the image data and to further process the extracted information. Accordingly, image analysis system 154 may include a digital computing device 162 (a controller) with a processor to perform data manipulation, and a memory to store instructions and data, among others. The instructions may direct the processing and extraction of information from the image data.

Image 160 (and/or corresponding image data) may include identifying information 164 for each cell set. The identifying information may include or correspond to code images 166, which correspond to the carrier codes and identify cell sets connected to the different carrier classes 120, 122, 124. The identifying information also may include a cell-association area and/or detection area 168 defined by each carrier. The detection area, as used herein, is a region of the carrier from which cell data is collected and used, generally for detecting an effect (or no effect) of modulators. The position of the cell-association/detection area may be defined, for example, relative to the perimeter of each carrier, relative to the coding regions, based on the area's optical properties (such as transparency), and/or the like. Cells belonging to each cell set may be inferred from image data based on proximity to the position of the cell-association area defined by each carrier.

Image 160 (and/or corresponding image data) also may include response information 170 for each cell set. The response information may include signal data corresponding to a signal(s) sensed for each cell set. The signal data may be derived from the image (or image data) by image analysis system 154. The signal data may be determined, for example, by defining an area of the image occupied by each cell set, masking a portion of the image substantially complementary to the area, and collecting/selecting signal data from the unmasked portion corresponding to the area. Signal data may be compared to control values/signals or to expected values, among others, to determine an effect, if any, of each potential modulator on the cell sets. Furthermore, effects on different cell sets may be compared with one another to define a selectivity of a potential modulator on a subset of the cell sets, such as a selective cytotoxicity on disease cells with a disease phenotype (e.g., loss of growth control) relative to normal cells lacking the disease phenotype. Further aspects of measuring signals and detecting effects are described, for example, in Sections VI and XI, and elsewhere in the present teachings.

II. Primary Cells

The assays of the present teachings may be performed with any suitable cells, but may be performed advantageously with primary cells. Primary cells, as used herein, are cells that have been isolated recently from a multi-cellular organism, such as an animal (particularly, a person or people) or plant. Alternatively, or in addition, primary cells are cells placed in assays without in vitro transformation and/or immortalization (e.g., by transfection, infection, treatment with a chemical mutagen, repeated passage, etc.), after their isolation and before the start of assays.

Cells are considered to be isolated recently, as used herein, based on the amount of time elapsed and/or the number of cell divisions that have occurred since isolation from an organism. Any suitable amount of time may have elapsed, for example, if the primary cells were frozen between isolation and use. However, in some embodiments, the cells may have been isolated within about six months, one month, one week, or one day of their use in assays. In some examples, the cells may have not been frozen at any time between their isolation and use in cells assays or may have been frozen at least once. Alternatively, or in addition, cells that have isolated recently may have been cultured after isolation for less than about ten, five, or two cell divisions.

Primary cells may be isolated from any suitable human or nonhuman organisms with any suitable genetic background and at any suitable developmental stage. Exemplary genetic backgrounds and/or developmental stages may include, but are not limited to, wild-type, mutant, transgenic, chimeric zygote, morula, blastula, embryo, fetus, newborn, juvenile, adolescent, adult, senior, male, female, and/or the like.

The primary cells of each sample may be isolated from one tissue and/or organ or may be isolated from a plurality of tissues or organs (or from two more distinct and/or spaced sites within the same tissue or organ). Examples of tissues/organs from which primary cells may be isolated include blood, brain, kidney, liver, lung, heart, muscle, nerve, tendon, vessel, bone, eye, skin, subcutaneous tissue, visceral tissue, and/or pancreas, among others.

The primary cells may be of one type or a mixture of two or more types. Exemplary types of primary cells include blood stem cells, B- and T-lymphocytes, red blood cells, neutrophils, eosinophils, mast cells, granulocytes, megakaryocytes, macrophages, adipose cells (pre-adipocytes and/or adipocytes), glial cells, astrocytes, neuroblasts, neurons, skeletal myoblasts or myotubes, smooth muscle myoblasts, cardiac myoblasts, fibroblasts, osteoblasts, osteocytes, endocrine cells, exocrine cells, islet cells, endothelial cells, keratinocytes, chondrocytes, cells derived from endoderm, mesoderm, or ectoderm, and/or extraembryonic derivatives, such as trophoblasts, among others. Types of primary cells further may include embryonic and adult stem cells. Mixtures of types of primary cells may occur naturally, may be produced by the isolation procedure, and/or may be formed after isolation, among others.

The primary cells may be isolated by any suitable technique from any suitable site of the body. Exemplary techniques include drawing fluid (e.g., blood, lymph, extracellular fluid, tissue/organ fluid, mucus, urine, tears, semen, etc.) from the body (such as with a needle, catheter, or pipet, among others) or from a tissue explant, collecting this fluid as it leaves the body through an opening (naturally or through an injury, incision, etc.), and/or explanting a generally solid tissue sample (such as by cutting, coring, scraping, etc.), among others.

In some examples, the cells from a tissue sample may be aggregated, that is, isolated with a majority of the cells disposed in one or more aggregates (masses) of cells, with each mass or aggregate including at least about twenty cells held together by an extracellular matrix and/or through cell-cell interactions, among others. Accordingly, aggregated cells may be isolated and used in assays without disaggregation. Alternatively, primary cells may be isolated in a substantially nonaggregated (dispersed) configuration, in which a majority of the cells are not included in aggregates. In some examples, the primary cells may be isolated in an aggregated configuration and disaggregated somewhat or completely before cells are selected and placed in assays, that is, before the primary cells are connected to carriers. Disaggregation may be performed by any suitable technique, including a chemical treatment (e.g., with a digestive enzyme (such as trypsin) and/or an altered salt composition), mechanical disruption (e.g., by vortexing, douncing, etc.), and/or the like.

In some cases, primary cells may be co-cultured with other cells, including primary or nonprimary cells, from the same or a different tissue, and from the same or a different animal or species. Such co-culturing may be performed for any suitable reason, including ease of isolation, the provision of physical support and/or cofactors, and/or the induction of certain behaviors or responses (such as synapse formation, fusion, assembly into multi-cellular structures, etc.), among others. Exemplary co-cultured cells may include neurons and glial cells, among others.

The primary cells may be engineered/modified before and/or after their isolation by introduction of foreign nucleic acids, for example, nucleic acids introduced by transfection (infection, lipofection, injection, etc.), or the cells may not be modified substantially by foreign nucleic acids. The foreign nucleic acids may be stably integrated into the genomes of the cells, and/or they may be extrachromosomal. Extrachromosomal nucleic acids may be replicating, for example, as episomes, or may be nonreplicating. The foreign nucleic acids may express a protein. Examples of expressed proteins include cell-surface receptors, such as ion channel-linked receptors (neurotransmitter receptors), G protein-coupled receptors (GPCRs), enzyme-linked receptors (such as tyrosine kinases, serine-threonine kinases, phosphatases, etc.), and/or the like. Other receptors may include nuclear receptors, such as receptors for steroid hormones, thyroid hormones, vitamin D, retinoids, and/or the like. Alternatively, or in addition, the expressed proteins may include transcription factors (for example, AP-1, SP1, NF-κB, etc.), cell cycle regulators (such as cyclins and cyclin dependent kinases), proteins involved in signaling cascades (protein kinase C, Janus kinases, ERK kinases, MAP kinases, protein kinase A, PI3-kinase, ras, etc.), cytoskeletal proteins (such as actins, tubulins, intermediate filament proteins, and/or associated proteins), transporters, ion channels, extracellular matrix proteins, enzymes, and/or so on.

Primary cells may carry reporter genes introduced before and/or after isolation of the primary cells. In some examples, the reporter genes may be introduced after connection to carriers. Reporter genes may include regulatory sequences that provide a regulated transcriptional response. Such regulatory sequences may include promoters, enhancers, and/or target elements that respond to a particular signaling pathway, transcription factor, or set of transcription factors. For example, regulatory sequences may include nuclear receptor response elements, cyclic AMP response elements, NFAT response elements, interferon response elements, and/or the like. Reporter genes express reporter RNAs and/or reporter proteins. Expression levels of the reporter RNAs and/or proteins may be measured to as a signal relating an aspect of primary cells. Reporter proteins may include enzymes (for example, beta-galactosidase, chloramphenicol acetyltransferase, glucuronidase, luciferase, and/or so on), and/or optically detectable proteins (such as green fluorescent protein and/or its yellow, red, orange, and/or blue derivative, among others). Further aspects of transfecting cells after their connection to carriers are included in the patent applications listed above in the Cross-References, which are incorporated herein by reference, particularly U.S. patent application Ser. No. 10/382,818, filed Mar. 5, 2003.

Primary cells may include cells that are alive or dead, fixed or unfixed, and/or cultured or directly isolated from an organism, among others. Living primary cells may measure binding and/or phenotypic responses, and dead primary cells may be used as targets to measure binding and/or effects on activity of a molecular target of the cells, among others. Dead primary cells may be fixed using a fixative (such as an organic solvent, paraformaldehyde, glutaraldehyde, picric acid, and/or the like) or may be left unfixed.

The primary cells may provide targets for testing potential modulators. The term “target” is defined broadly, to include the primary cells themselves, components thereof, and/or activities associated with the primary cells, among others. Primary cells and targets that may be included in the assays of the present teachings are described further in the patents and patent applications listed above in the Cross-References, which are incorporated herein by reference, particularly U.S. patent application Ser. No. 10/120,900, filed Apr. 10, 2002.

Ill. Carriers

Primary cells and/or their components may be connected to carriers. The carriers generally comprise populations of relatively small carriers, termed microcarriers or particles. The particles may be of one type (generally lacking a code) or may be of two or more distinguishable types (or classes), for example, distinguishable at least in part by a detectable code, particularly an optically detectable code.

The carriers generally may have any suitable size, shape, and composition. Preferred properties are determined by the application. For example, preferred properties are determined in part by what the carriers support, with carriers preferably being larger or at least a few times larger than the molecules, organelles, viruses, cells (e.g., primary cells), and so on that they support. In some cases, the carriers may carry a code but may be too small to carry cells, so that they may be particles that function as labels disposed inside (e.g., by internalization or microinjection, among others), in the membrane, and/or connected to the outside of the cells. These labels may be smaller or at least about a few times smaller than the cells (e.g., primary cells), organelles, and so on that they label and/or that internalize them. Preferred properties also are determined in part by the detection method, with carriers preferably being (at least for optical detection) larger than the wavelength of light but smaller than the field of view. Preferred carrier sizes for sample support (that is, sizes for particles and/or microcarriers) range between about ten microns and about five millimeters. In some examples, the carriers may have a maximum characteristic dimension (generally, their length) that is less than about 2 millimeters and greater than the average diameter of primary cells connected to the carriers. Preferred carrier sizes for labels between about 10 nm and about one micron. Exemplary carrier geometries include generally planar, spherical, elliptical, and/or cylindrical, among others.

The code generally comprises any mechanism capable of distinguishing different carriers. The code may relate to overall features of the carriers. These features may include carrier size, shape, and composition. Alternatively, or in addition, the code may relate to subfeatures of the carriers. These subfeatures may be positional and/or nonpositional, meaning that the code is based on the presence, identities, amounts, and/or properties of materials at different positions in the carrier and/or at potentially the same position in the carrier, respectively. These positions may be random and/or predefined. Exemplary positional codes may include positioning different amounts and/or types of materials at different positions in or on a carrier, for example, at spots, lines, concentric circles, and the like. These positional codes may be read by determining the identities, amounts, and/or other properties of the code materials at each code position, for example, by measuring intensity as a function of position. Exemplary nonpositional codes may include using at least two different materials, potentially at the same position, where the materials differ in absorption, color (i.e., excitation and/or emission spectrum), intrinsic polarization, and/or any other measurably distinct property or characteristic. These nonpositional codes may be read by determining the presence and/or other properties of signals from the different materials, for example, by measuring intensity as a function of wavelength. In each case, the amounts, positions, and/or values may be relative or absolute. Moreover, different types of codes may be combined to form yet other types of codes.

Further aspects of carriers and coded carriers that may be included in the systems of the present teachings are described in more detail in the patent applications identified above in the Cross-References, which are incorporated herein by reference, particularly U.S. patent application Ser. No. 09/694,077, filed Oct. 19, 2000; U.S. patent application Ser. No. 10/120,900, filed Apr. 10, 2002; U.S. patent application Ser. No. 10/273,605, filed Oct. 18, 2002; and U.S. patent application Ser. No. 10/713,866, filed Nov. 14, 2003.

IV. Potential Modulators

Primary cells may be exposed to a potential modulator or a library of potential modulators to test the effect or activity of the potential modulator on the primary cells. A potential modulator, as used herein, is any condition, substance, and/or mixture that might have, or is expected to have, an effect on the cells or their components. The potential modulator may be a physical modulator, a chemical modulator, and/or biological modulator, among others. Furthermore, the potential modulator (or a set of potential modulators) may be a modulator(s) with unknown clinical effect or known clinical effect. A modulator with unknown clinical effect, as used herein, means that the modulator has not been tested in clinical trials, that is, the response of humans to the modulator has not been tested systematically, at least for the indication currently being investigated. By contrast, a modulator with known clinical effect, as used herein, means that systematic tests have been performed to measure how humans respond to administration of the modulator, at least for the indication currently being investigated.

Physical modulators include any physical state to which the primary cells are exposed. The physical state may be, for example, the temperature and/or pressure of an examination site holding the primary cells, and/or an amount or quality of light (electromagnetic radiation) at the site. Alternatively, or in addition, the physical modulator may relate to an electric field, magnetic field, and/or particle radiation to which the primary cells are exposed, among others.

Chemical modulators include any substance or composition to which the primary cells are exposed. The substance/composition may be a chemical compound, hydrogen ions (pH), ionic strength, and/or fluid composition, among others.

Biological modulators include any biological agent to which the primary cells are exposed. The biological agent may include cells, viruses, cell vesicles, organelles, cell extracts, and/or the like.

Primary cells may be assayed using a library of potential modulators to test, the effect of each library member on the cells. A library generally comprises a collection of two or more different members. These members may be chemical/biological modulators in the form of molecules, ligands, compounds, transfection materials, receptors, antibodies, cells, viruses, tissues, and/or cell extracts, among others, related by any suitable or desired common characteristic. This common characteristic may be “type.” Thus, the library may comprise a collection of two or more different compounds, two or more different cells, two or more different antibodies, two or more different nucleic acids, two or more different ligands, two or more different receptors, or two or more different viruses, among others. This common characteristic also may be “function.” Thus, the library may comprise a collection of two or more binding partners (e.g., ligands and/or receptors), agonists, or antagonists, among others, independent of type.

Library members that are potential modulators may be produced and/or otherwise generated or collected by any suitable mechanism, including chemical synthesis in vitro, enzymatic synthesis in vitro, and/or biosynthesis in a cell or organism. Chemically and/or enzymatically synthesized libraries may include libraries of compounds, such as synthetic oligonucleotides (DNA, RNA, peptide nucleic acids, and/or mixtures or modified derivatives thereof), small molecules (for example, about 100 Da to 10 KDa), peptides, carbohydrates, lipids, and/or so on. Such chemically and/or enzymatically synthesized libraries may be formed by directed synthesis of individual library members, combinatorial synthesis of sets of library members, and/or random synthetic approaches. Library members produced by biosynthesis may include libraries of plasmids, complementary DNAs, genomic DNAs, RNAs, viruses, phages, cells, proteins, peptides, carbohydrates, lipids, extracellular matrices, cell lysates, cell mixtures, and/or materials secreted from cells, among others.

Further aspects of modulators and libraries of modulators are described in the patent applications listed above under Cross-References, which are incorporated herein by reference, particularly U.S. patent application Ser. No. 10/120,900, filed Apr. 10, 2002.

V. Vessels

Any suitable vessels may be used to hold primary cells, fluid, modulators, assay reagents, and/or carriers. The vessels may be used at any suitable time, including before, during, and/or after connection of cells to carriers, exposure of cells to potential modulators, addition of assay reagents, and/or detection of effects of modulators on cells. The same or different vessels may be used during connection, exposure, and/or detection. Exemplary vessels that may be suitable include beakers, test tubes, microcentrifuge tubes, dishes (such as petri dishes), capillary tubes, and/or microplates, among others.

Microplates generally comprise sample holders having a frame and a plurality of individual sample wells disposed in the frame for holding a corresponding number of samples and/or fluid volumes. Microplates may be rectangular in shape, generally with cylindrical, hexahedral, or frustoconical wells arranged in pre-defined arrays (for example, rectangular or other geometric arrays), enabling the sample holder to be used with standard microplate equipment, such as handlers, washers, and/or readers, among others.

Microplates may be designed and manufactured as desired, for example, in accordance with industry standards published by the Microplate Standards Development Committee of the Society for Biomolecular Screening. The industry-standard frame has a major dimension X of 127.76 millimeters (mm)±0.5 mm, a minor dimension Y of 85.48 mm±0.5 mm, and a height Z of 14.35 mm±0.76 mm, although other dimensions are possible. In addition, the rigidity of an industry-standard microplate is specified such that at any point along the sidewalls, the differential displacement is no greater than 0.50 mm between an applied load of 0.10 kilograms (kg) and an applied load of 1.00 kg. The frame may include a base configured to facilitate handling and/or stacking, and a notch configured to facilitate receiving a cover. The following table shows three preferred industry-standard well configurations, where D_{c} is the distance from the left edge of the plate to the center of first well column, and DR is the distance from the top edge of the plate to the center of the first well row:

| | | | | | Density |
|---|---|---|---|---|---|
| Number | Arrangement | D_{C} | D_{R} | Pitch (mm) | (/mm²) |
| of Wells | of Wells | (mm) | (mm) | Between Wells | of Wells |
| 96 | 8 × 12 | 14.38 | 11.24 | 9 | 1/81 |
| 384 | 16 × 24 | 12.13 | 8.99 | 4.5 | 4/81 |
| 1536 | 32 × 48 | 11.005 | 7.865 | 2.25 | 16/81 |

The color and material of the microplate may be selected to facilitate particular applications, for example, as shown in the following table:

| Application | Preferred Plate Color and/or Material |
|---|---|
| DNA Libraries/Cell Culture | Clear Polystyrene |
| Fluorescence | Black Polystyrene |
| Luminescence | White Polystyrene |
| High Temperature/Solvent | Clear Polypropylene |
| Resistant | |
| Adherent Cell Assays | Clear Bottom (Black or White) |
| DNA Quantization | Clear UV Transparent |

In exemplary embodiments, the microplate may be configured for optical detection of assay results from below the microplate, that is, configured to detect light received below the microplate from the bottom of the microplate wells. Accordingly, the bottom may be substantially transparent to visible, UV, and/or IR light. Furthermore, the bottom may be thin enough to achieve optical resolution of results from individual sub-wells and/or individual cells disposed within the wells and/or sub-wells. Exemplary thicknesses include less than or equal to about 2 mm, 1 mm, 0.5 mm, and/or 0.25 mm, among others, including 0.9 mm and 0.17 mm, among others.

Carriers and cells may be placed in sample holders configured to provide adjustable fluid communication. For example, the sample holders may be configured as microplates with wells having inner and outer walls of different height to form sub-wells within the wells. The level of fluid in the wells thus can be adjusted to permit cells in sub-wells of a well to be in fluidic isolation or in fluidic communication at different times during an assay. Further aspects of sample holders, particularly sample holder that permit adjustable fluid communication, are described in more detail in the patent applications listed above in the Cross-References, which are incorporated herein by reference, particularly U.S. patent application Ser. No. 10/282,940, filed Oct. 28, 2002; and U.S. Provisional Patent Application Ser. No. 60/585,150, filed Jul. 2, 2004.

VI. Detection Systems and Cell Assays

Detection systems (and cells/reagents) of the present teachings may be configured to facilitate performing any suitable cell assays. Sample signals (or characteristics) from samples (e.g., sets of primary cells and/or a compartment or fluid in which the cells are/were disposed) may be measured using any suitable detection system(s) at any suitable time(s) during/after an assay.

Sample signals, codes, and/or other measured quantities may be determined using any suitable detection mechanism of a detection system. The measured quantities generally comprise any measurable, countable, and/or comparable property or aspect of interest. The detection mechanism may measure by spectroscopic, hydrodynamic, imaging, magnetic, and/or electrical methods, among others, especially those adaptable to high-throughput analysis of multiple samples. Alternatively, or in addition, the detection mechanism may include the eye(s) or a user, to measure a signal by visual inspection. Measured quantities may be reported quantitatively and/or qualitatively, as appropriate. Measured quantities may include presence or absence, or relative and/or absolute amounts, among others.

The detection mechanism may be configured to perform optical detection, such as with a light source and a detector. The light source may provide trans-illumination through samples to the detector, for example, to measure absorbance, scattering, photoluminescence, or microscopic pattern (bright field, dark field, DIC, Nomarski, phase contrast, etc.), among others. Alternatively, the light source may be disposed on the same side of the samples as the detector, for example, both below a microplate, to provide epi-illumination. Epi-illumination may be used, for example, to measure photoluminescence, such as fluorescence or phosphorescence, among others. Alternatively, the light source may be disposed at any other suitable angle(s) or position relative to the detector and samples to perform, for example, measurements of total internal reflection. In some embodiments, such as measurements of sample bio-, chemi-, or electroluminescence, a light source may not be required for optical measurements. Exemplary apparatus for detecting sample signals may include any suitable detection devices and/or optics, such as a point sensor, an array of sensors (such as a CCD array), an electrode, a microscope, a film scanner, a fiber optic bundle, and/or a plate reader, among others.

The sample signals may correspond to any suitable portion or all of one or more samples. Generally, a sample signal(s) represents data about a set of cells (either collectively, or for an individual cell or subcellular component/region of the set), generally a set of cells connected to one carrier or disposed in a separate compartment/sub-compartment of a sample holder. Alternatively, a sample signal may correspond to an averaged signal detected from a plurality of cell sets, for example, to identify the presence of a rare positive set among many cell sets in a library screen.

The detection system also may include a controller that performs data processing. The controller may perform, for example, data manipulation, image analysis, background subtraction, comparison of values, identification of sets of cells based on associated codes and/or positions, etc. Accordingly, in some examples, the controller may facilitate detecting an effect by processing sample data, control data, code data, position data, image data, etc.

The present teachings provide assays with primary cells. The assays may measure any suitable aspect of the cells. Exemplary assays include differentiation assays that measure the changes in differentiation markers, cytotoxicity assays to measure cell death, assays that measured an undesired effect (such as changes in growth rate, metabolism, etc.) of a potential modulator, signal transduction assays, reporter gene assays with endogeous or exogenous reporter genes, polymerase chain reaction assays to measure DNA and/or RNA (generally preceded by treatment with a lysis reagent (e.g., detergent) to disrupt cells), receptor activation assays, genotyping assays, and/or the like. Exemplary aspects include a characteristic of the cells in the presence and absence of one or more potential modulators, to determine an effect, if any, of the presence of the potential modulators on the cells. Other exemplary aspects include measuring a characteristic of the cell without testing the effect of a potential modulator.

Any suitable cell characteristics (aspects) may be measured (detected). Exemplary characteristics may include the presence/absence, amount, activity, shape, size, movement, function, integrity, and/or appearance of cells, cell organelles, cell components, and/or associated reagents, among others. Exemplary cell characteristics may include cell growth (e.g., progression through the cell cycle, increase in cell number, arrest in the cell cycle, etc.), cell death/cytotoxicity (e.g., number and/or ratio of cells that are alive versus those that are dead), and/or reporter gene activity, among others. Further aspects of detection systems (also termed measurement systems), cell assays, and cell characteristics that may be measured in the assays of the present teachings are described in the patent applications listed above in the Cross-References, which are incorporated herein by reference, particularly Ser. No. 10/120,900, filed Apr. 10, 2002.

VII. Selecting Primary Cells

Any suitable primary cells may be selected for use in assays of the present teachings. The primary cells may be selected at any suitable time, after any suitable manipulation, and from any suitable organism(s), particularly one or more human subjects. The human subjects may be selected based on any suitable criteria, as described further below.

Cells may be selected for use at any suitable time after their isolation. The cells may be selected immediately after isolation from an organism, after growth of the cells in culture for one or more cell divisions, after the cells have been frozen, and/or the like.

Cells may be selected after any suitable manipulation has been performed after their isolation. Exemplary manipulations include disaggregation, incubation in growth medium, passaging (growth and dilution), freezing, counting, centrifugation, fixation, sorting, selective or nonselective killing, cell-type enrichment, addition of other cells, infection with biological agents (viruses, bacteria, etc.), transfection, treatment with growth factors, and/or the like. However, these manipulations generally do not immortalize and/or transform the cells.

A set of primary cells (a sample) may be isolated from a human subject, that is, from any person (or people). Each sample may be isolated from one person or may be a mixture of primary cells from two or more people. The person may be of any suitable age, health, disease state, ethnic background, gender, weight, etc.

In some examples, a plurality of distinct samples for an assay may be isolated from the same human subject, such as from different body sites, tissues, and/or cell types, among others, of the subject. In some examples, the subject may be a disease patient, that is, a person known to have a particular disease or medical condition. In some example, the disease patient may have a disease that selectively affects a subset of the regions, tissues, and/or cell types isolated from the patient, for example, to screen modulators for a selective or nonselective effect (such as cytotoxicity or an undesired effect) on affected (or nonaffected) regions, tissues, and/or cell types.

A disease patient (a human subject having a disease) may have any suitable disease (or medical condition). Exemplary diseases that may be suitable include allergies (e.g., rhinitis, sinusitis, hives, etc.), Alzheimer's Disease, arthritis (e.g., ankylosing spondylitis, fibromyalgia, gout, lupus, osteoarthritis, psoriatic arthritis, reactive arthritis, rheumatoid arthritis), baldness, cancer (e.g., brain tumor, bladder cancer, breast cancer, cervical cancer, colon cancer, liver cancer, lung cancer, pancreatic cancer, prostate cancer, skin cancer, testicular cancer, etc.), high cholesterol, chronic pain, colds, flu, Crohn's Disease, psychiatric problems (e.g., bipolar disorder, dysthymia, depression, panic disorder, post traumatic stress disorder, seasonal affective disorder (SAD), stress, psychoses, diabetes (e.g., diabetes mellitus, diabetes insipidus, etc.), digestion problems (e.g., appendicitis, ulcerative colitis, GERD (Heartburn, Acid Reflux), hemorrhoids, irritable bowel syndrome (IBS), lactose intolerance, etc.), eye disease (e.g., cataracts, glaucoma, macular degeneration, conjunctivitis, Sjogren's Syndrome, etc.), cardiovascular disease (e.g., angina, atherosclerosis, congenital heart disease, heart attack, etc.), hepatitis (e.g., cirrhosis, cryoglobulinemia, Hepatitis A/B/C, lichen planus, etc.), hypertension, HIV infection (e.g., acquired immunodeficiency syndrome), lung disease (e.g., asthma, emphysema, pneumonia, severe acute respiratory syndrome (SARS)), osteoporosis, skin disease (e.g., acne, actinic keratosis, atopic dermatitis (eczema), boils, psoriasis, dermatitis, rosacea, scleroderma, shingles, warts, etc.), thyroid disease (e.g., hyperthyroidism, hypothyroidism, etc.), urologic disease (e.g., cystinuria, interstitial cystitis, kidney stones, prostatitis, etc.), weight control diseases (e.g., anorexia nervosa, bulimia, obesity, etc.), central nervous system diseases, and/or the like.

Primary cells from a disease patient may be diseased (having a disease phenotype) or nondiseased (having a nondiseased or normal phenotype). A disease phenotype, as used herein, is any aspect of the cells that correlates with the presence of a corresponding disease. For example, a cancer patient may have diseased cells with a disease phenotype of reduced contact inhibition, altered cell morphology, increased chromosomal abnormalities, a particular genetic mutation, altered cell cycle progression, and/or the like. Furthermore, a diabetes patient may have diseased cells with a disease phenotype of insulin nonresponsiveness or reduced responsiveness. A nondiseased phenotype, as used herein, is the substantial absence of a particular disease phenotype of interest in (nondiseased) primary cells. Accordingly, nondiseased primary cells may serve as normal and/or control cells for experiments with one or more sets of diseased cells.

In some examples, a plurality of samples for an assay may be isolated from a plurality of human subjects, such as from the same region or tissue and/or from different regions or tissues, among others, of the human subjects. The human subjects may include one or more human subjects substantially lacking a selected disease (representing normal or control subjects), and/or one or more human subjects (disease patients) having the selected disease (representing disease samples and/or control samples from a nondiseased region of the subject). In some cases, the human subjects having the selected disease may have distinct forms of the disease (such as different stages of a type of cancer), which may be identified and/or categorized according to any suitable criteria. The samples may include one or more samples from each of two or more forms/stages/severities of the disease. Further aspects of selecting cell samples from disease patients are described in Example 1 of Section XIV.

Any suitable number of sets of primary cells may be selected for an assay. In some examples, at least about two, ten, twenty, fifty, or one-hundred sets of primary cells may be selected from a respective at least two, ten, twenty, fifty, or one-hundred human subjects. In some examples, the human subjects may be selected to represent the entire human population or a particular subset of the human population (such as adult males, North American adults, children, an at-risk subset, etc.)

VIII. Connecting Primary Cells to Carriers and/or Compartment Surfaces

Primary cells may be connected to carriers and/or compartment surfaces, generally after the cells have been selected for use in assays. The cells may be connected by any suitable association mechanism(s) including covalent and/or noncovalent associations.

Any suitable number of primary cells may be connected to carriers and/or compartment surfaces and at any suitable density. In some examples, the number of primary cells connected to each carrier or compartment surface region (generally, the surface of a sub-compartment) may be about 1-10,000, less than about 2,000, and/or greater than about twenty or fifty. The density of the primary cells may be substantially uniform or nonuniform over the surface of each carrier and/or sub-compartment. In some examples, the primary cells may be selectively connected to a subset of the carrier and/or compartment surfaces (e.g., a single face, opposing faces, and/or a detection area of the carrier, or a single sub-compartment of a compartment, among others), so that the primary cells are concentrated on a side(s), face(s), or other surface region of the carrier or compartment surface. Furthermore, the primary cells may be connected so that they are disposed in the particles, on the particles, and/or about the particles (or sub-compartment surface). The primary cells may be connected at any suitable level of confluence or may be incubated after connection, and generally before detection of a signal, to achieve this level of confluence. In some examples, the cells may be incubated after connection, and before detecting, until they have reached a preselected level of confluence. The cells may be connected at, and/or incubated until, any level of confluence has been achieved. For example, at least about one-half of the cells may be clustered to provide at least about 10%, 20%, 50%, or 80% coverage of a surface region of a carrier or the support surface of a sub-compartment. Alternatively, or in addition, the cells may be disposed so that at least about one-half of the primary cells of a carrier or sub-compartment (one-half based on the number of connected primary cells) are spaced from another cell by less than the average diameter of the primary cells. The spacing of adjacent cells is measured as the perimeter-to-perimeter separation, if any, of adjacent cells at their position of closest cell-cell approach. Alternatively, or in addition, the primary cells may be disposed so that at least about one-half of the primary cells of a carrier or a sub-compartment (at least one-half of the number of connected primary cells) are in physical contact with another cell, that is, substantially touching another cell. In other examples, any of these levels of confluence may be for cells of the carriers disposed in a detection area of the carriers, from which the cell signal is detected.

Further aspects of connecting cells to carriers and sub-compartment surfaces are included in the patent applications listed above in the Cross-References, which are incorporated herein by reference, particularly U.S. patent application Ser. No. 10/282,940, filed Oct. 28, 2002; U.S. patent application Ser. No. 10/407,630, filed Apr. 4, 2003; and U.S. Provisional Patent Application Ser. No. 60/585,150, filed Jul. 2, 2004; and U.S. patent application Ser. No. 10/901,942, filed Jul. 28, 2004.

IX. Placing Primary Cells in Compartments

Primary cells may be placed in compartments for exposure to modulators at any suitable time(s) during an assay. The cells may be connected to particles before or after they are placed in the compartments, and/or the cells may be connected to the compartments after they are placed in the compartments.

Cells may be connected to one or more particles after the step of placing the cells and particles in a compartment. In particular, particles may be added to the compartment first, before the cells are added. The compartment and the particles may be configured so that the cells added after the particles are selectively connected to the particles relative to the support surface of the compartment. For example, the compartment may have a hydrophobic or other nonadherent surface. Accordingly, cells that do not connect to the particles, after they are placed in the compartment (e.g., cells resting on the support surface of the compartment), may be removed selectively after the step of connection, to create a locally increased density of the cells adjacent the particles relative to the support surface. The cells then may be assayed in the compartment.

Sets of cells connected to particles may be placed into the same compartment, such as a microplate well, as a mixture of the sets, or individually. For example, each set of cells may be connected to one or more particles having a different code, so that the cells can be identified when the different coded particles are mixed.

A single set of cells connected to one or more particles may be placed into a compartment for a “singleplexed” analysis of the cells. Accordingly, the one or more particles may lack a code. In some embodiments, the single set of cells connected to a plurality of particles may be dispensed to each of a set of sub-compartments of a compartment.

The primary cells and/or their carriers may be placed in one or more compartments of a sample holder to form an array. The array generally comprises any set of two or more samples (primary cell sets) that have distinct positions in a sample holder. The distinct positions may be within a shared volume of fluid (that is, in the same compartment), such as arbitrary/random positions in a well of a microplate. Accordingly, the distinct positions may be addressable by including a potential modulator in the shared volume. The array thus may be a nonpositional, or arbitrarily positioned array, in which at least some or all samples of the array have an arbitrary position relative to each other. Alternatively, or in addition, the distinct positions may be disposed in fluidic isolation, such as separate wells or sub-wells of a microplate. Accordingly, the samples may be identifiable at least partially by their positions in the array.

A nonpositional array may be formed by combining samples that have been associated with distinguishable coded carriers. Each sample may be associated separately with coded carriers having one or a set of distinguishable codes. Each resulting sample-carrier assembly then may have a distinct class determined by the associated sample and identifiable by reading the distinguishable code(s). Accordingly, an array of identifiable samples may be formed by combining/mixing distinct classes of sample-carrier assemblies. Such an array generally includes at least two distinct samples associated with carriers of different types (for example, having distinct codes). In some embodiments, sample may be distributed in a partially positional array in which each sample is included in a nonpositional array that has a position within a higher order array, for example, formed by the wells of a microplate. In such a partially positional array, a sample may be identified by an associated code in combination with a position within the higher order array.

A positional array may be formed by positioning samples relative to each other and/or relative to a fixed structure. Samples positioned relative to each other are identifiable based at least partially on these relative positions. For example, samples may be distributed in a spaced array on a substrate, using an asymmetrical arrangement for the purpose of orientation. Alternatively, or in addition, samples may be distributed in a spaced array on a substrate having a landmark structure, such as a recess, a protrusion, a marking, and/or so on. In either case, an individual sample may be identified based on the position of the individual sample relative to other samples and/or relative to the landmark structure.

In some examples, the fluidic relationship between samples may be adjusted during the course of an assay. For example the samples and their carriers, may be placed in different sub-wells of a well, so that the samples are in fluidic isolation. The fluidically isolated samples may be treated with different potential modulators (and/or different reagents) and then disposed in fluidic communication by adding fluid to an appropriate level to the sub-wells. The samples then may be exposed to the same reagent and/or potential modulator. Alternatively, the samples may be exposed to a modulator/reagent in fluidic communication and then exposed to another modulator/reagent in fluidic isolation.

Exemplary arrays and methods of forming arrays, including mixing carriers, dispensing carriers, placing carriers into vessels, and placing cells into sub-compartments of sample holders, are described in more detail in the patent applications identified above in the Cross-References, which are incorporated herein by reference, particularly Ser. No. 10/120,900, filed Apr. 10, 2002; U.S. patent application Ser. No. 10/282,940, filed Oct. 28, 2002; U.S. Provisional Patent Application Ser. No. 60/585,150, filed Jul. 2, 2004; and U.S. patent application Ser. No. 10/901,942, filed Jul. 28, 2004.

X. Exposing Cells to Potential Modulators

Primary cells may be exposed to one or more potential modulators during an assay. The cells may be exposed to any suitable modulators for any suitable period of time during the assay. In some examples, the period of time may be less than one hour, less than one day, at least about one day, or at least about three days, among others. The exposure may be continuous, transient, periodic, sporadic, etc.

The modulator may include only one potential modulator or at least two or more potential modulators. The two or more modulators may be exposed to the cells as a mixture of two or more substance. The mixture may permit the cells to be exposed to a greater number of substances, to screen, for example, for a relatively rare substance that produces an effect on the cells. Alternatively, or in addition, the mixture may permit screening for a combination of substances that produces a selected effect on the cells. The selected effect may include, for example, improved specificity of the effect, a synergy of the substances, reduced cytotoxicity, a reduction in undesired effects, etc. In some examples, the cells may be exposed to a potential modulator and a candidate agent, to screen, for example, for a candidate agent that increases efficacy, reduces cytotoxicity, and/or the like. For example, the potential modulator may have an unknown clinical effect, and the candidate agent may have a known clinical effect, or both may have known or unknown clinical effects.

Cells may be exposed to potential modulators at any suitable concentration(s). In some examples, set of cells may be exposed to different concentrations of the same potential modulator(s), for example, to determine a dose-response relationship for the potential modulator(s) on the cells.

XI. Detecting Effects of Potential Modulators

The assay systems of the present teachings may detect effects, if any, of potential modulators on sets of primary cells. The effects generally comprise and/or result from any physical binding or other interaction of a potential modulator with a set of cells and/or a component(s) thereof. Detected effects generally represent effects that are measurable as significant changes, within the sensitivity/variability of the assay system and the particular type of effect being measured, typically produced by exposure to potential modulators relative to the absence of the potential modulators. Accordingly, in many cases, detection of no effect may represent an effect too weak to measure or to consider significant in the particular assay system.

Effects may be detected selectively for exposure of the sets of primary cells to a potential modulator. Selective detection of effects, as used herein, refers to detecting one or more effects that are correlated with one or more individual cell sets, rather a global effect on all the sets of cells exposed to a nonselective potential modulator. For example, observation that all sets of cells are killed by a particular potential modulator, without identification of any of the sets of cells individually, is nonselective detection rather than selective detection.

Physical binding generally represents any detectable association of a potential modulator with a set of cells or components thereof. Binding may include specific binding, that is, binding to a specific binding partner to the exclusion of binding to most other moieties. Specific binding can be characterized by a dissociation constant or coefficient (alternatively termed an affinity or binding constant or coefficient). Generally, dissociation constants for specific binding range from 10⁻⁴ M to 10⁻¹² M and lower, and preferred dissociation constants for specific binding range from 10⁻⁸ or 10⁻⁹ M to 10⁻¹² M and lower.

Detectable changes in one or more characteristics (aspects) of a set of cells may be determined by the nature of the target provided by the cells. Molecular targets, such as proteins, enzymes, peptides, and nucleic acids, among others, may interact with a potential modulator to produce detectable changes in conformation and/or activity, among others. Such changes may be detectable by spectroscopic methods (such as absorbance, fluorescence, reflectance, scattering, etc.), surface plasmon resonance, nuclear magnetic resonance, enzyme assay, and/or so on.

Detectable effects of modulators, other than those measured as physical binding, may include an aspect related to a phenotypic effect on cells. Phenotypic effects generally represent any change in the cells produced by the modulator only if the cells are alive. Exemplary phenotypic changes include changes in level, localization/movement, modification, morphology, structure, conformation, and/or activity of any cellular component, complex, structure, organelle, and/or whole cells, among others. Examples of levels of cellular components may include levels of total RNA, tRNAs, specific mRNAs, and/or hnRNAs, among others; levels of proteins, peptides, glycoproteins, proteoglycans, and/or reporter proteins (such as beta-galactosidase, luciferase, green fluorescent protein, chloramphenicol acetyltransferase, and/or the like), among others; levels of lipids, such as specific phosphoinositides and forms of cholesterol; and/or the like. Examples of localization may include localization of a component or complex to a cellular organelle or region, such as the nucleus, cytoplasm, Golgi apparatus, lysosomes, nuclear membrane, endoplasmic reticulum, endosomes, cell membranes, cell-surface, extracellular matrix, etc. Accordingly, changes in localization may include transfer of a component between any two or more of these structures or movement of one of these structures itself. Examples of modification may include phosphorylation, acetylation, methylation, glycosylation, amidation, gamma-carboxylation, ubiquitination, farnesylation, and/or the like. Conformation or structure may include primary, secondary, tertiary, or quaternary structural aspects. Exemplary changes in conformation or structure may be mediated by cleavage enzymes, ligases, isomerases, epimerases, gyrases, topoisomerases, molecular interactions, etc. Examples of morphologies may include shape of cells, organelles, and membranes, among others. Examples of activities include enzyme activities, electrical activities (such as ion currents or membrane voltages), and/or the like.

Detecting effects generally includes detecting (sensing) one or more sample signals corresponding to each set of cells. The sample signals may be detected from a compartment in which the cells are/were disposed. Detecting signals from a compartment, as used herein, detects signals from any portion or all of the compartment including cells, particles, fluid in the compartment, and/or the surface of the compartment. Before, during, or after detecting sample signals, a code may be read and/or the position of a set of cells determined, to permit the set to be identified based on the code and/or the position. The steps of detecting sample signal and identifying the set of cells may be performed in any order, and each step may be performed selectively on specific carriers/sets of cells/positions. For example, in some cases, set of cells may be identified only if they exhibit a preselected sample signal. Alternatively, sample signal may be detected only for cell sets that have a specific code(s) or position in a sample holder. In some examples, sample signals and codes may be determined by image analysis from image data of the cells and/or particles using any suitable image analysis algorithms and/or parameters, such as described in U.S. patent application Ser. No. 10/282,904, filed Oct. 28, 2002, which is incorporated herein by reference.

In some examples, detecting a signal from a sample may include identifying particles on which the density of cells equal or exceeds a desired level of confluence, and detecting a signal from the identified particles to the exclusion of detecting a signal from other particles for which the density of cells is less than the desired level of confluence.

Further aspects of cell characteristics (aspects), phenotypes, interactions, and detecting effects are described in more detail in the patent applications identified above in the Cross-References, which are incorporated herein by reference, particularly U.S. patent application Ser. No. 10/120,900, filed Apr. 10, 2002; Ser. No. 10/282,904, filed Oct. 28, 2002; and Ser. No. 10/444,573, filed May 23, 2003.

XII. Selecting Modulators and/or Human Subjects Based on Detected Effects

The systems of the present teachings permit particular modulators and/or human subjects to be selected based on detected effects of the modulators and/or on the human subjects.

A plurality of modulators may be screened for an effect on sets of primary cells, and a subset of one or more of the modulators may be selected based on the effect, to prioritize the selected subset relative to the other modulators. In some examples, selection may include stratifying the plurality of modulators into more than two groups. For examples, the plurality of modulators may be stratified into three or more groups, based on any suitable criteria, such as expected success in clinical trials (e.g., for treatment of a disease or condition under investigation), strength of effect, specificity of effect, level of undesired effects, level of toxicity, and/or the like. This stratification of the modulators into three or more groups may be for any suitable purpose, such as prioritization for further analysis (study).

Selection of the subset of modulators may be for any suitable purpose. For example, the subset of modulators may be selected for further study, for exclusion from further studies, etc. Further analysis, as used herein, may include any additional tests performed with the subset of modulators. Exemplary further analysis may include administration to one or more human subjects such as in clinical trials, administration to one or more nonhuman animals (animal studies), additional in vitro assays (such as structure-activity studies, derivatization studies, testing other phenotypes, toxicity studies, etc.), and/or the like.

A subset of one or more human subjects may be selected based on the detected effects. The subset of human subjects may represent a subset of donors that contributed sets of primary cells to an assay. The subset of human subjects may be selected for any suitable purpose. Exemplary purposes include exclusion of their set(s) of cell from further study, contribution of one or more additional sets of cells for further study, treatment with a modulator that produced an effect on sets of cells from the human subjects, and/or the like. In some examples, a human subject may be selected from a set of human subjects that provided sets of primary cells, and a particular modulator out of set of potential modulators may be selected for administration to the human subject, based on a detected effect of the particular modulator on the human subject.

XIII. Databases

The systems of the present teachings may provide databases of detected effects (and/or no effects) produced by potential modulators on human subjects. These databases may include any suitable information about the human subjects (e.g., genotypic information), to permit analysis of data in the databases, for example, to correlate the information about the human subjects to their response to potential modulators. Accordingly, these databases may guide selection of potential modulators for treating other disease patients (e.g., patients outside the database), for derivatization, for design of additional assays, etc.

Multiplexed analysis according to the present teachings optionally may be used to determine structure-activity relationships and/or other information for a library of potential modulators, such as chemical compounds. This analysis may generate data on the effects of potential modulators on primary cells in a novel way, resulting in unique databases of information. In particular, multiplexed technology may be used to collapse aspects of both a primary screen and a secondary screen of modulators. (The primary screen generally corresponding to a potency screen of all the modulators, and the secondary screen corresponds to a specificity screen of a subset of the modulators that are more potent.) As a result, multiplexed technology may collect additional information, such as specificity, from an entire library of potential modulators rather than merely from those members of the library that are identified as “hits” in the primary screen. Therefore, multiplexed technology may generate a comprehensive database suitable not only for determination of structure-activity relationships, but also to guide rational design of future screens. These unique databases may not only be more comprehensive than databases compiled using nonmultiplexed technology, but they also may be used in ways that these other databases cannot.

Databases generated with primary cells according to the present teachings may be collected, stored, manipulated, and/or displayed using any suitable mechanism. Typically, elements of the databases will be treated as ordered arrays, that is, as collections of identifying indicia and results. Exemplary databases and a graphical method for displaying and analyzing them are described in U.S. patent application Ser. No. 10/444,573, filed May 23, 2003, which is incorporated herein by reference.

XIV. Examples

The following examples describe selected aspects and embodiments of the present teachings, particularly exemplary assays performed with primary cells connected to coded carriers and data from these assays. These examples are included for illustration and are not intended to limit or define the entire scope of the present teachings.

### Example 1

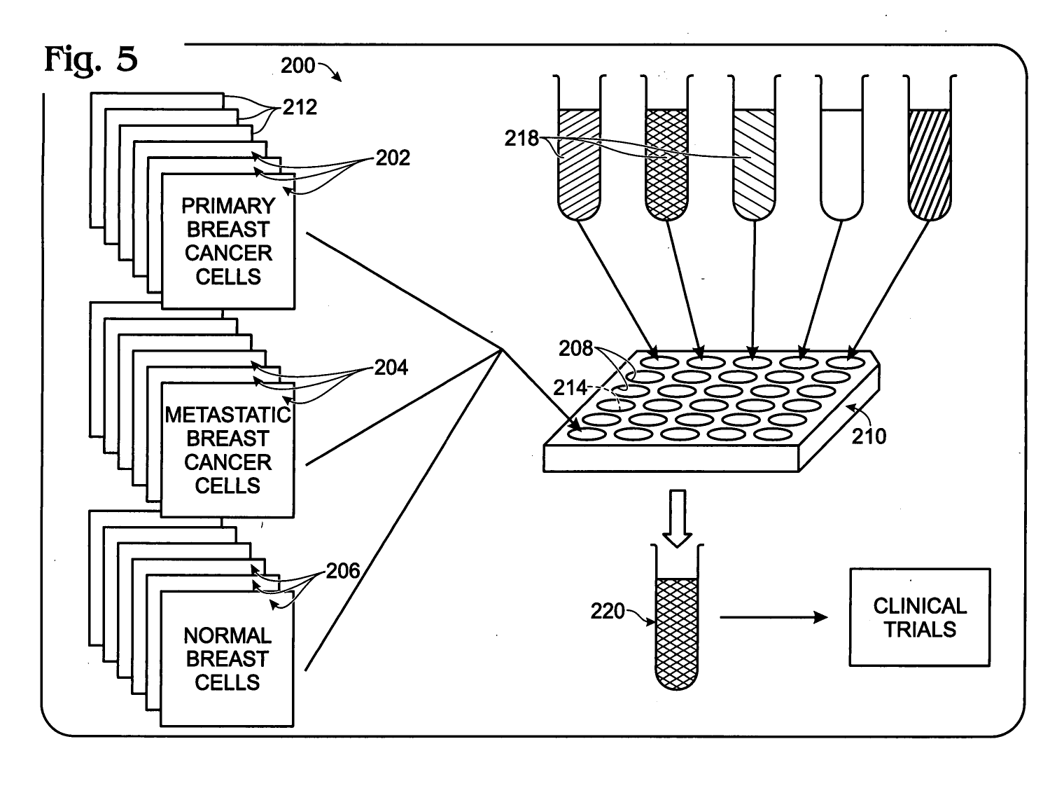

This example describes how primary cells may be used to stratify (classify and/or prioritize) human subjects and/or compounds in vitro for further study (such as clinical trials, animal studies, etc.); see FIG. 5.

FIG. 5 shows a flowchart of an exemplary method 200 of assaying primary cells. In brief, sets of primary cells may be isolated from human subjects having or lacking a particular disease. For example, in the present illustration, sets of primary breast cancer cells 202, metastatic breast cancer cells 204, and normal (noncancerous) breast cells 206 are isolated from cancer patients and/or normal (control) human subjects.

Members of each set of cells may be placed at a localized high density within one or more compartments, such as wells 208 of a microplate 210. For example, members of each set of cells may be connected to one or more particles 212 to concentrate the members adjacent the particles relative to the entire support surface 214 of a well. Each set of cells may be attached to or otherwise associated with a different type of carrier (e.g., having a different code(s)). The identity (disease stage/type, patient of origin, etc.) of the cells on a given carrier thus may be determined by identifying the type of the carrier (e.g., by reading the code of the carrier). Accordingly, a plurality of the sets of primary cells may be assayed together in a shared fluid volume, in this case, in the same well. In some examples, members of each set of cells may be assayed without distinguishable carriers, such as by disposing the members of a set (with or without carriers) at an identifiable position within a well, such as a sub-well formed within a divided well. Further aspects of wells, sub-wells, and method of using wells and sub-wells for performing assays are included in the following patent applications, which are incorporated herein by reference: U.S. patent application Ser. No. 10/282,940, filed Oct. 28, 2002; and U.S. Provisional Patent Application Ser. No. 60/585,150, filed Jul. 2, 2004.

The sets of primary cells may be exposed to a panel of compounds 218 by adding each compound to the compartment(s) in which the sets of primary cells are disposed. For example, when the sets of primary cells are disposed in a shared fluid volume (e.g., in a divided well or connected to coded particles and disposed in the same well), a compound may be added to (included in) the shared volume to expose the sets of cells to the compound at the same time under a common set of conditions in the shared volume. During and/or after exposure to the compounds, signals may be measured from the sets of primary cells, so that an effect, if any, of each compound on each set of primary cells may be detected selectively, that is, so that each set can be identified. Accordingly, this approach may identify selective, nontoxic, and/or more effective compounds, among others, for use in subsequent studies (such as clinical trials), such as the compound indicated at 220. In some examples, this approach may provide an indication of the population variability of an effect (if any) produced by each compound, by assaying the effect of the compound on a plurality of human subjects.

### Example 2

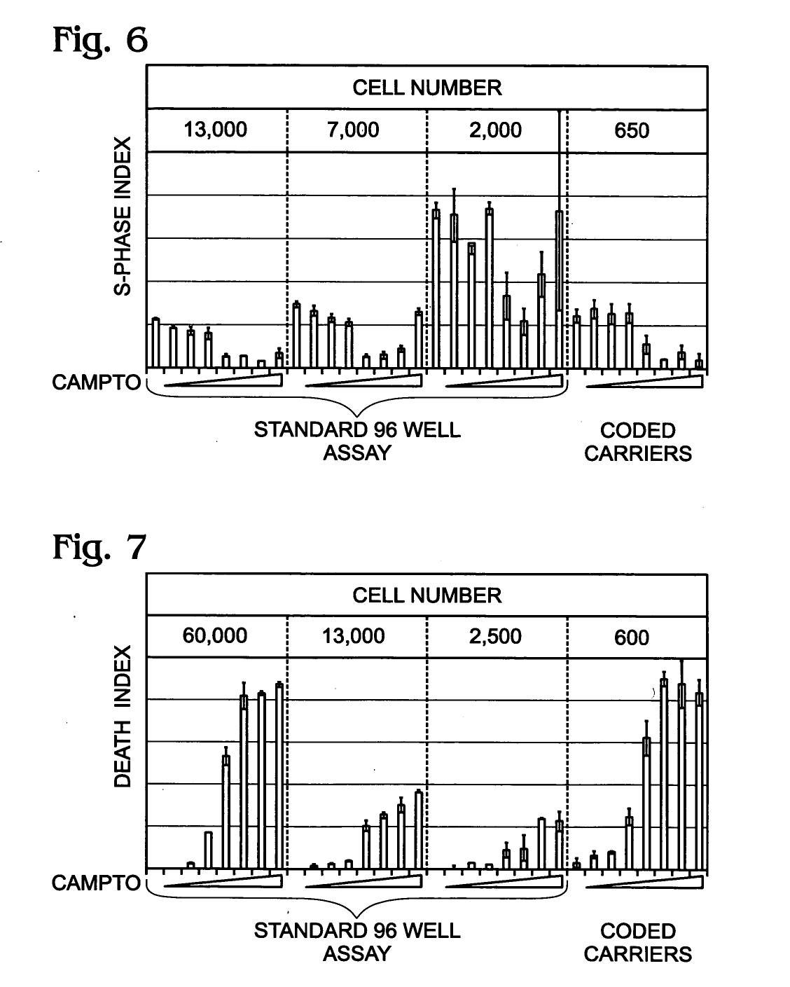

This example describes how multiplexed analysis using coded carriers may be used to assay the effects of camptothecin on the proliferative response of primary human renal proximal tubule epithelial (HRPTE) cells, in accordance with aspects of the present teachings; see FIG. 6.

Camptothecin is an alkaloid from a plant. Camptothecin is believed to inhibit topoisomerase 1. This biological activity has generated medical interest in camptothecin as a potential anti-tumor chemotherapy agent, particularly for cancers such as colorectal cancers that involve overexpression of topoisomerase 1.

FIG. 6 shows results from an assay in which the effects of camptothecin (“Campto”) on HRPTE proliferation are studied by adding the compound at various concentrations to sample containers in which the HRPTE cells are growing. Specifically, HRPTE cells are grown directly in wells of a standard 96-well microplate, to three different cell densities (indicated as the stated number of cells), or on coded carriers that optionally may be placed in wells of the microplate. An “S-phase index” (e.g., the fraction of cells in the S-phase of the cell cycle) is used as a measure of cell proliferation. Here, the S-phase index generally decreases with increasing camptothecin concentration (indicated by the triangles disposed below the X-axis), showing that camptothecin inhibits DNA synthesis necessary for cell division, and that this inhibition increases with increasing camptothecin concentration. Significantly, the results obtained using only about 650 cells attached to coded carriers are comparable to the results obtained using about 13,000 cells attached directly to microplate wells, and better than those obtained using 7,000 or 2,000 cells attached directly to microplate wells. Thus, in some embodiments, a coded-carrier system may be used to obtain data comparable to data obtained with a standard microplate assay, using only about 5% as many cells, or less. This reduction may be particularly significant in assays on primary cells, because primary cells typically are difficult to obtain and maintain.

### Example 3

This example describes how multiplexed analysis using coded carriers may be used to study the cytotoxicity response of HRPTE cells to staurosporine, in accordance with aspects of the present teachings; see FIG. 7.

Staurosporine is a natural product originally isolated from the bacterium Streptomyces staurosporeus. Staurosporine is believed to inhibit kinase enzymes, including but not limited to protein kinase C (PKC), by interfering with binding of adenosine triphosphate (ATP). This biological activity has generated medical interest in staurosporine as a potential anti-tumor chemotherapy agent, because aberrations in kinase activity may underlie the explosive cell growth associated with cancer.

FIG. 7 shows results from an assay in which the cytotoxicity response of HRPTE cells to staurosporine is studied by adding the compound at various concentrations (indicated by the triangles disposed below the X-axis) to sample containers in which the cells are growing. Specifically, HRPTE cells are grown directly attached to wells of a standard 96-well microplate, or on coded carriers that optionally may be placed in wells of the microplate. A “death index” (e.g., the fraction of observed cells that are dead) may be used as a measure of cytotoxicity. The death index may be computed using any suitable mechanism, such as a fluorescence-based “live/dead assay.” In an exemplary version of the assay, live cells are labeled green using a membrane-permeant calcein AM, which is cleaved by esterases in live cells to yield cytoplasmic green fluorescence, and dead cells are labeled red using a membrane-impermeant ethidium homodimer-1, which passes through the compromised membranes of dead cells to label nucleic acids with red fluorescence. Here, the death index (i.e., the fraction of dead cells) generally increases with increasing staurosporine concentration. Significantly, the results obtained using only about 600 cells grown on coded carriers are comparable to the results obtained using about 60,000 cells grown directly in microplate wells, and better than those obtained using 13,000 or 2,500 cells attached directly to microplate wells. Thus, in some embodiments, a coded-carrier system may be used to obtain data comparable to data obtained with a standard microplate assay, using only about 1% as many cells (or less). This reduction may be particularly significant in assays on primary cells, because primary cells typically are difficult to obtain and maintain.

### Example 4

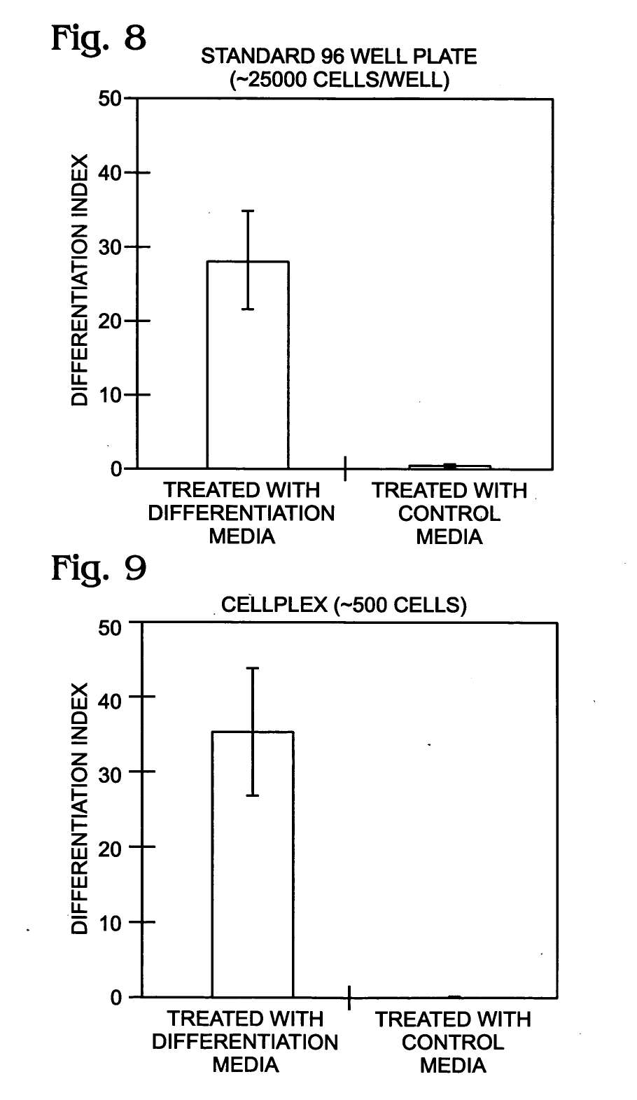

This example describes adipocyte differentiation assays and/or PPARγ activity assays performed with primary cells attached to microplate wells or coded carriers, particularly coded carriers disposed in a shared fluid volume, in accordance with aspects of the present teachings; see FIGS. 8-12. The assays may be used, for example, to select/prioritize compounds for further study as candidates for treatment of type II diabetes (and/or for administration to type II diabetic patients, among others), to select type II diabetic patients for treatment with a compound, and/or to identify PPARγ modulators and/or modulators of adipocyte differentiation, among others.

Adult onset diabetes (type II) represents a major proportion (about 90%) of the diagnosed cases of diabetes and is a chronic disease with substantial health and monetary implications. Type II diabetes is non-insulin dependent and apparently related to obesity, high cholesterol, and high blood pressure. Target cells of diabetic patients (generally, muscle, fat, and liver cells) bind insulin normally, but inefficiently transport glucose into the cells as a downstream consequence of this binding. Accordingly, glucose accumulates to high levels in the blood, resulting in a glucose toxicity that can lead to retinopathy (blindness); neuropathy (nerve damage), which may lead to foot ulcers, gangrene, and/or amputations, among others; kidney damage, which may lead to dialysis; and/or cardiovascular disease, among others.

Type II diabetes can be treated with agonists of PPARγ. PPARγ (peroxisome proliferation associated receptor-gamma) is a nuclear hormone receptor that functions as a transcription factor. PPARγ positively autoregulates its own expression. In particular, PPARγ is typically expressed at low levels and is slowly induced after its activation with an agonist. PPARγ activation has been shown to result in differentiation of pre-adipocytes (such as fibroblasts) to adipocytes (fat cells). This differentiation may be visualized readily and scored by staining lipid droplets that accumulate in the cytoplasm of the adipocytes during and/or after their differentiation. Furthermore, this adipocyte differentiation and/or activation of PPARγ may be followed by measuring the expression levels (RNA and/or protein) of PPARγ target genes (direct or indirect targets), such as PEPCK (phosphoenolpyruvate carboxykinsae) and/or FABP4 (fatty acid binding protein 4), among others. The differentiation of pre-adipocytes into fat cells by the activation of PPARγ, and/or induction of adipocyte markers, may be used as surrogate assays for identifying PPARγ agonists for use in treating type II diabetes and/or other medical conditions affected by PPARγ activity.

A set of thiazolidinediones, the “glitazones,” have been used successfully as a clinical treatment to alleviate symptoms of type II diabetes. The glitazones have been shown to function as PPARγ agonists and to induce adipocyte differentiation in vitro. However, the glitazones may not be effective for many type II diabetic patients due to population variability and/or may produce undesired side effects such as increases in the number and/or size of fat cells. Accordingly, new technology is needed to prioritize lead compounds for further study using in vitro analysis of primary cells, particularly to select compounds with less population variability. Furthermore, new technology is needed to select particular patients for administration of particular compounds and/or to select treatment regimens based on in vitro analysis of primary cells.

FIGS. 8 and 9 show the results of adipocyte differentiation assays treated with a cocktail of inducers or no inducer. FIG. 8 shows results from differentiation assays performed with a relatively large number of cells disposed in separate wells of a standard 96-well microplate. FIG. 9 shows results from differentiation assays performed by multiplexed analysis using coded carriers and about 1/50 as many cells as the assay of FIG. 8. Primary cells for the assays were pre-adipocytes supplied by Cambrex Corporation. The adipocytes were placed at (or grown to) about 80% confluency in the separate wells (FIG. 8; about 25,000 cells/well) or on a detection area of the coded carriers (FIG. 9; about 500 cells/well). Cells were treated with a control medium or a differentiation medium (Cambrex) also including a mixture of dexamethasone, IBMX, indomethacin, and insulin. Cells were stained for lipid with AdipoRed™ (Cambrex) after an eight-day incubation with their respective media, to provide a measure of adipbcyte differentiation. Comparable differentiation efficiencies (“differentiation index”) were obtained within each set of experiments. Furthermore, about 50-fold fewer cells may be used with coded carriers relative to microplate wells without carriers. The density/confluence of the cells also was found to be important for efficient differentiation in other experiments not presented here. In particular, at levels of confluence less than about 80%, differentiation dropped off dramatically. Accordingly, the use of carriers allows this high confluence to cells to be achieved with a relatively small number of cells.

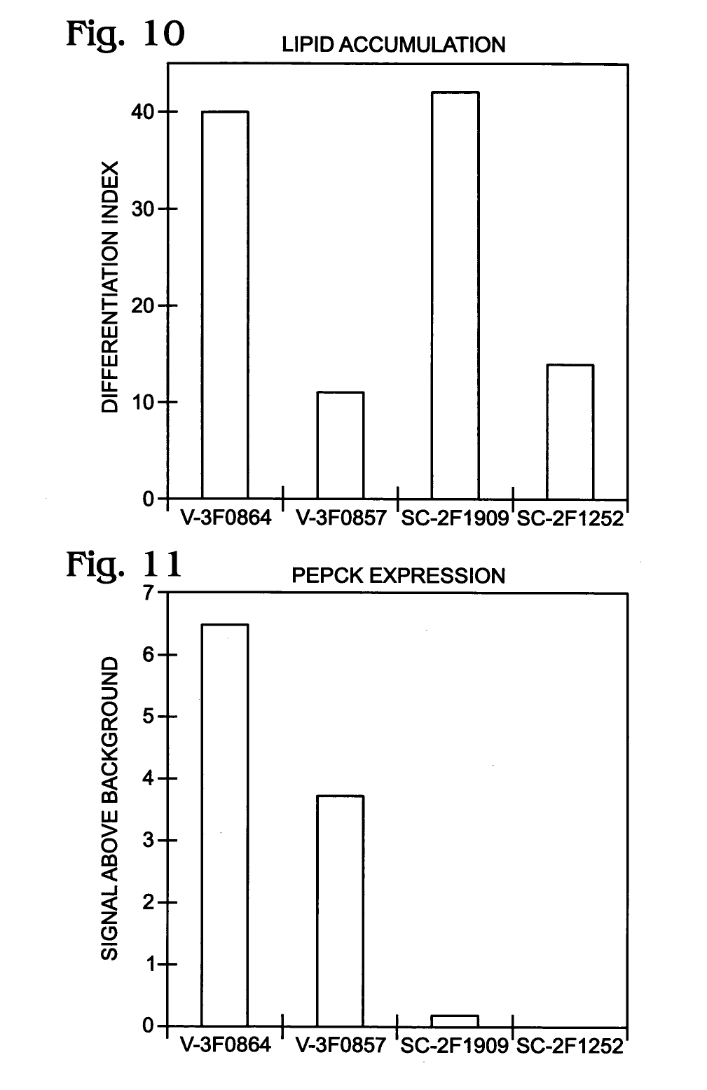

FIGS. 10 and 11 are graphs showing the results of adipocyte differentiation assays and PPARγ activity assays, respectively, performed in multiplex on sets of primary cells (samples) from the same four human subjects by using coded carriers disposed in a shared fluid volume. FIG. 10 shows results obtained by measuring lipid accumulation to provide a differentiation index for each sample. FIG. 11 shows results obtained from corresponding samples by measuring PEPCK expression (“signal above background”) with an anti-PEPCK antibody supplied by Dr. Darryl Granner of Vanderbilt University. Staining was via indirect immunofluorescence. Cells were counterstained with a nuclear dye (Hoechst) for data normalization, here and in other assays. Here and elsewhere in the Examples, sample signals were determined by analysis of image data using image analysis algorithms and parameters, for example, generally as described in U.S. patent application Ser. No. 10/282,904, filed Oct. 28, 2002, which is incorporated herein by reference. Each sample of primary cells corresponds to pre-adipocytes isolated from visceral tissue (samples starting with the letter “V”) or isolated from subcutaneous tissue (samples starting with the letters “SC”) and were supplied by Cambrex. Each sample has an anonymous identifier (3F0864, 3F0857, 2F1909, or 2F1252) for tracking the sample. Each sample was connected to a distinct class of coded carriers (having a distinct code) to permit identification of the sample after combining the different classes of carrier and samples in a shared volume. Furthermore, each sample was connected to a surface region of the corresponding class of carriers so that the primary cells were disposed at a desired degree of confluence (about 80% coverage of the surface region of each carrier). In some examples, the surface region may be all or a portion of a planar or rilled face of a carrier. The different classes of carriers and their connected sets of primary cells then were placed in two microplate wells (one well for the data of each graph) and exposed to troglitazone in a shared volume of fluid over the course of multiple days. Lipid accumulation and PEPCK expression were then measured with the samples still disposed in the same microplate wells. Since the samples producing data for each graph were disposed in the same microplate well, experimental variations in reagent addition and assay conditions are substantially eliminated between the samples the same well. Accordingly, differences in lipid accumulation or PEPCK expression among samples apparently represent real sample-to-sample variation for differentiation and PEPCK induction.

The results presented in FIGS. 10 and 11 may be summarized as follows. Both the visceral samples and the subcutaneous samples show variation in differentiation based on the differentiation index, suggesting substantial donor (and population) variability for differentiation responses (at least about four-fold). In these data, neither tissue source shows a substantial difference in average differentiation index. In contrast, PEPCK expression shows a marked difference between visceral and subcutaneous samples, with each visceral sample providing dramatically increased PEPCK over each subcutaneous sample. PEPCK expression therefore does not correlate with lipid accumulation in these subcutaneous samples. Overall, based on data from this assay, subject 3F0864 may be selected as the best candidate of the subjects for a strong response to troglitazone treatment, subject 3F0857 as a candidate for a moderate response to troglitazone treatment. The other subjects may be expected to exhibit a weaker response to troglitazone treatment, particularly in subcutaneous tissue.

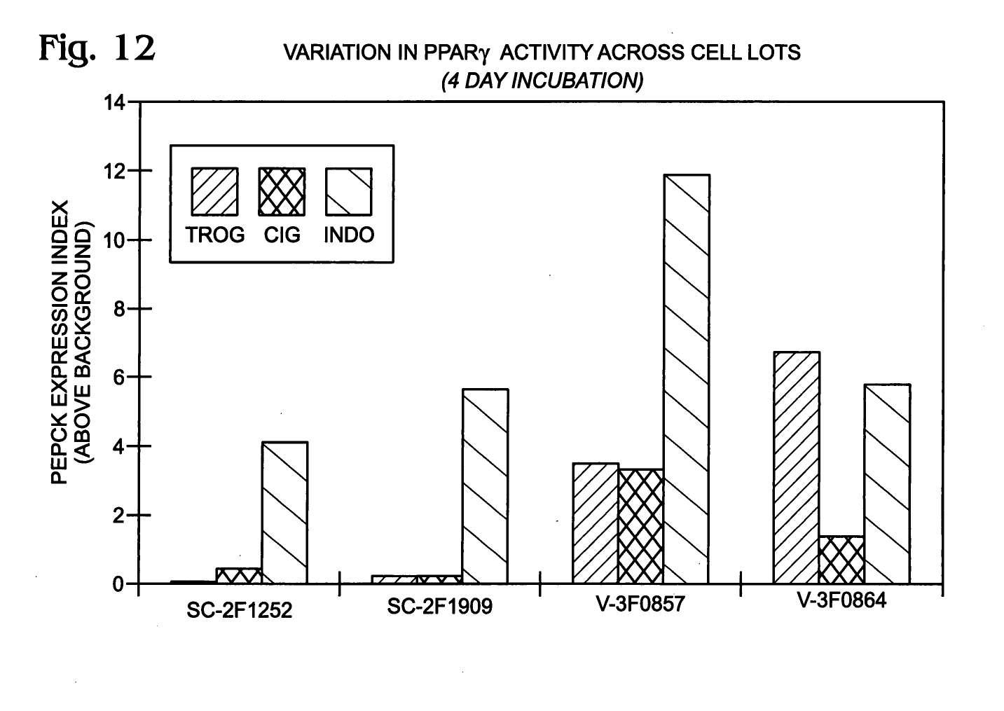

FIG. 12 is a graph showing the results of PPARγ activity assays that compare the effects of different PPARγ agonists on primary cells from the same samples (human subjects and tissue sources) as FIGS. 10 and 11. The samples were connected to different classes of coded carriers, as described above, and distributed as mixtures to three microplate wells. The carriers and their connected samples in each microplate well were exposed, in a shared fluid volume to troglitazone (TROG), ciglitazone (CIG), or indomethacin (INDO) for four days to test induction of PEPCK expression as a measure of PPARγ activity.

The results of FIG. 12 can be summarized as follows. Indomethacin produces a relatively strong induction of PEPCK expression in each the donor samples. By contrast, troglitazone produces a moderate and variable induction of PEPCK, and ciglitazone produces a weak and variable induction. Accordingly, if these inducers were potential modulators of unknown clinical effect, they may be prioritized for further study according to the strength and/or uniformity of response they induce in a plurality of subject samples, for example, in the following order: (1) indomethacin, (2) troglitazone, and (3) ciglitazone. However, any suitable criteria may be used to prioritize potential modulators of unknown clinical effect for further study, with the result that the order of priority may vary, depending on the criterion or criteria used in the prioritization.

Overall, the studies of this example may be summarized as follows. (1) Human primary pre-adipocytes remain viable and differentiate on coded carriers of the present teachings over the course of about 4-12 days. Accordingly, these assays can be miniaturized to permit a larger number of assays with fewer primary cells. (2) PEPCK antibody works as a reporter for PPARγ activity. (3) These and other data show that ciglitazone and troglitazone preferentially activate PEPCK expression in visceral samples. (4) Lipid accumulation does not always correlate with PEPCK expression

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

1. A method of testing potential modulators of primary cells, comprising:
selecting sets of primary cells isolated from at least one human subject;
exposing the sets of primary cells to a potential modulator of unknown clinical effect in a shared volume of fluid; and
detecting an effect selectively, if any, of the step of exposing on each of the sets of primary cells.

2. The method of claim 1, wherein the step of selecting includes selecting sets of primary cells isolated from at least two human subjects.

3. The method of claim 1, wherein the step of selecting sets of primary cells includes selecting a set of primary cells isolated from at least two patients having a disease and at least one human subject substantially lacking the disease.

4. The method of claim 1, wherein the step of selecting sets of primary cells includes a step of selecting sets of primary cells isolated from patients with different forms of the same disease.

5. The method of claim 1, wherein the step of selecting sets of primary cells includes a step of selecting sets of primary cells from diabetes patients.

6. The method of claim 5, wherein the step of detecting an effect includes a step of measuring an aspect related to differentiation of the sets of primary cells into adipocytes.

7. The method of claim 1, wherein the step of selecting sets of primary cells includes a step of selecting sets of primary cells from cancer patients.

8. The method of claim 1, wherein the steps of exposing and detecting are performed a plurality of times with different potential modulators.

9. The method of claim 8, the step of detecting producing data, the method further comprising a step of selecting a subset of the potential modulators for administration to people based on the data.

10. The method of claim 1, wherein the steps of exposing and detecting are performed a plurality of times with different concentrations of the same potential modulator.

11. The method of claim 1, the step of detecting producing data, the method further comprising a step of selecting a subset of the human subjects to be treated with the potential modulator based on the data.

12. The method of claim 1, wherein the primary cells have a disease phenotype that is distinct from a normal phenotype of the primary cells, and wherein the effect is a change in the disease phenotype.

13. The method of claim 1, wherein the step of detecting includes a step of detecting an undesired effect.

14. The method of claim 1, further comprising a step of connecting members of each of the sets of primary cells to a different type of particle prior to the step of exposing, such that the members of each set can be identified by determining the type of particle to which they are connected.

15. The method of claim 14, wherein the step of connecting members includes a step of connecting members of each of the sets of primary cells to one or more particles having a code.

16. The method of claim 1, further comprising the steps of:
disposing members of each of the sets of primary cells in separate compartments so that that the members of different sets are in fluidic isolation from each other; and
adding fluid to at least some of the compartments so that the fluid reaches a level that brings the compartments into fluidic communication, before or concurrent with the step of detecting.

17. The method of claim 1, wherein the steps of exposing and detecting are performed with the sets of primary cells disposed in one or more wells of a 96-well microplate.

18. The method of claim 1, wherein the step of selecting includes a step of selecting sets of primary cells isolated from a plurality of tissues of the at least one human subject.

19. A method of prioritizing potential modulators for further study, comprising:
selecting at least ten sets of primary cells isolated from at least ten human subjects;
exposing the at least ten sets of primary cells to a plurality of potential modulators of unknown clinical effect;
detecting one or more effects of exposing each of the at least ten sets of primary cells to the plurality of potential modulators; and
selecting a subset of the potential modulators for further analysis based on the effects.

20. The method of claim 19, wherein the steps of selecting, exposing, and detecting are performed with at least fifty sets of primary cells.

21. The method of claim 19, wherein the step of selecting at least ten sets of primary cells includes a step of selecting a plurality of sets of primary cells isolated from human subjects having the same disease and another plurality of sets of primary cells isolated from human subjects substantially lacking the disease.

22. The method of claim 19, wherein the step of selecting at least ten sets of primary cells includes a step of selecting sets of primary cells isolated from patients with different forms of the same disease.

23. The method of claim 19, wherein the step of detecting one or more effects includes a step of measuring an aspect related to differentiation of the sets of primary cells into adipocytes.

24. The method of claim 19, the method further comprising a step of selecting a subset of the at least ten human subjects to be treated with the subset of the potential modulators based on the effects.

25. The method of claim 19, wherein the primary cells have a disease phenotype that is distinct from a normal phenotype of the primary cells, and wherein the effects are a change in the disease phenotype.

26. The method of claim 19, wherein the step of detecting includes a step of measuring a level of toxicity produced by the step of exposing on each of the at least ten sets of primary cells.

27. The method of claim 19, further comprising a step of connecting members of each of the at least ten sets of primary cells to a different type of particle prior to the step of exposing, such that the members of each set can be identified by determining the type of particle to which they are connected.

28. The method of claim 19, wherein the step of exposing includes a step of exposing the at least ten sets of primary cells to one or more of the potential modulators in a shared volume of fluid.

29. The method of claim 19, further comprising the steps of:
disposing members of each of the at least ten sets of primary cells in separate compartments so that that the members of different sets are in fluidic isolation from each other; and
adding fluid to at least some of the compartments so that the fluid reaches a level that brings the compartments into fluidic communication, before or concurrent with the step of detecting.

30. The method of claim 19, wherein the step of selecting a subset of the potential modulators includes a step of selecting the subset for studies of structure-activity relationships.

31. The method of claim 19, wherein the step of selecting a subset of the potential modulators includes a step of selecting the subset for animal studies.

32. The method of claim 19, wherein the step of selecting a subset of the potential modulators includes a step of selecting the subset for administration to people.

33. A method of testing potential modulators of primary cells, comprising:
selecting sets of primary cells isolated from a plurality of human subjects;
connecting each set to one or more particles;
exposing the sets of primary cells to a potential modulator while the sets are connected to the particles; and
detecting an effect, if any, of the step of exposing on each of the sets of primary cells.

34. The method of claim 33, wherein the step of exposing includes exposing the sets of primary cells to a potential modulator of unknown clinical effect.

35. The method of claim 33, the particles each having a code corresponding to one of the sets of primary cells, wherein the step of detecting an effect includes a step of reading the codes to identify the set of primary cells connected to each particle.

36. The method of claim 33, wherein the step of exposing is performed in a shared volume of fluid.

37. A system for testing potential modulators of primary cells, comprising:
sets of primary cells connected to particles, the sets being isolated from a plurality of human subjects;
one or more vessels holding the particles and the connected sets of primary cells; and
a detection system configured to detect for each set an effect, if any, of exposing the sets to a potential modulator.

38. The system of claim 37, wherein the one or more vessels include one or more microplates having wells, and wherein the sets are disposed in fluidic isolation from each other in different wells.

39. The system of claim 37, wherein the particles include codes, and wherein the codes are configured so that the sets can be identified by reading the codes of the particles to which the sets are connected.

40. The system of claim 37, wherein the detection system includes an imaging mechanism.

41. A system for testing potential modulators of primary cells, comprising:
means for exposing sets of primary cells to a potential modulator while the sets of primary cells are connected to particles; and
means for detecting an effect, if any, of the potential modulator on each of the sets of primary cells.
